(19) United States
(12) Patent Application Publication
(10) Pub. No.: US 2020/0031805 A1
(43) Pub. Date: Jan. 30, 2020

(54) ARYL HYDROCARBON RECEPTOR (AHR) MODULATOR COMPOUNDS

(71) Applicant: PHENEX PHARMACEUTICALS AG, Ludwigshafen (DE)

(72) Inventors: Ulrich DEUSCHLE, Speyer (DE); Christoph STEENECK, Heidelberg (DE); Michael ALBERS, Mannheim (DE); Thomas HOFFMANN, Speyer (DE)

(21) Appl. No.: 16/483,981

(22) PCT Filed: Feb. 21, 2018
(86) PCT No.: PCT/EP2018/054234

§ 371 Date: Aug. 6, 2019

(30) Foreign Application Priority Data
Feb. 21, 2017 (EP) 17000276.0

(51) Int. Cl. C07D 403/12; A61K 45/06; A61K 31/4155; A61K 31/4196; C07D 401/12; A61K 31/4439; A61K 31/404; C07D 405/12; C07D 209/08

(52) U.S. Cl. CPC C07D 403/12; A61K 45/06; A61K 31/4155; A61K 31/4196; C07D 209/08; A61K 31/4439; A61K 31/404; C07D 405/12; C07D 401/12

(57) ABSTRACT

The present invention relates to 6-amido-1H-indol-2-yl compounds which can act as aryl hydrocarbon receptor (AhR) modulators and, in particular, as AhR antagonists. The invention further relates to the use of the compounds for the treatment and/or prophylaxis of diseases and/or conditions through binding of said aryl hydrocarbon receptor by said compounds.

The present invention relates to compounds which can act as aryl hydrocarbon receptor (AhR) modulators and, in particular, as AhR antagonists. The invention further relates to the use of the compounds for the treatment and/or prophylaxis of diseases and/or conditions through binding of said aryl hydrocarbon receptor by said compounds.

The aryl hydrocarbon receptor (AhR) is a ligand-modulated transcription factor, belonging to the basic helix-loop-helix PAS (Per-Arnt-Sim homology domain) family, that is expressed in most tissues in mice and humans and known to mediate many of the toxicities of 2,3,7,8-Tetrachlorodibenzo-p-dioxin (TCDD) in mice. The AhR protein is localized in the cytoplasm of eukaryotic cells in complexes with HSP90 and other proteins. Binding of agonistic ligands, such as TCDD, leads to dissociation of AhR from the HSP90 containing complex, transport to the nucleus and association with its heterodimeric partner ARNT. This heterodimeric complex can bind to AhR response elements located in promoter regions of genes such as CYP1A1, CYP1B1, ALDH3A1, NQO1, UGT1A1 etc. and induces the transcription of such genes in case of very potent and efficacious AhR agonists, such as TODD.

By regulating the expression of genes involved in xenobiotic transformation (e.g. CYP1A1), the AhR plays a significant role in the detoxification of xenobiotic substances in liver and intestine, which are prominent locations of AhR expression. This activity might be underlying some of the described chemoprevention and tumor suppression effects exerted by AhR. On the other hand, CYP1A1 is known to metabolize some pro-cancerogens, such as benzo(a)pyrene into DNA reactive intermediates leading to mutagenesis and tumor formation (Murray et al. Nat Rev Cancer. 2014 December; 14(12):801-14; Safe et al Toxicol Sci. 2013 September; 135(1):1-16).

In mouse cancer models, knock-down of AhR typically resulted in decreased proliferation and/or invasion and migration of cancer cell lines and overexpression of constitutive active AhR results in vivo in enhanced stomach and liver cancers (Safe et al Toxicol Sci. 2013 September; 135(1):1-16).

The AhR is relatively strongly expressed in intestinal epithelial tissues, lung epithelium and skin. In these tissues the AhR expression is particularly high in cells of lymphoid origin such as T-cells, Dendritic Cells, Langerhans Cells, Macrophages, Mast cells etc. One possible function in these compartments is to integrate signals from the commensal microbiomes in the intestine, the lung and the skin, which are known to produce diverse mixtures of indolic AhR modulators that are thought to balance the responses of the immune system towards the microbiome (Bessede et al., Nature. 2014 Jul. 10; 511(7508):184-90, Zelante et al. Immunity. 2013 Aug. 22; 39(2):372-85, Romani et al., Eur J Immunol. 2014 November; 44(11):3192-200).

The expression of AhR has been found to be constitutive active in advanced human prostate cancer (Richmond et al., 2014, PLoS ONE 9(4): e95058), overexpressed in breast cancer (Li et al., Int J Clin Exp Pathol. 2014 Oct. 15; 7(11):7931) and pancreas cancer (Koliopanos et al., Oncogene. 2002 Sep. 5; 21(39):6059-70). Modulation of the AhR pathway activity by small molecule modulators might be beneficial for some of these devastating diseases with very limited treatment options.

In a recently published Patent Application US 2016/01752278 by the Trustees of Boston University, novel small molecule agents characterized as AhR modulators are being claimed for inhibiting cancer cell proliferation and tumor cell invasion and metastasis.

AhR modulators and in particular modulators with primarily antagonistic activities might be useful as medicaments for the treatment of solid tumors (e.g., pancreatic cancer, prostate cancer, breast cancer, colon cancer).

The problem underlying the present invention is to provide compounds which have a AhR-antagonistic activity and can be used in the treatment and/or prophylaxis of AhR-mediated diseases.

Said problem has been solved by a compound according to the following Formula (I), an enantiomer, diastereomer, tautomer, solvate, prodrug or pharmaceutical acceptable salt thereof

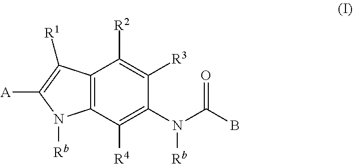

wherein
A is selected from 6- to 10-membered mono- or bicyclic aryl and 5- to 10-membered mono- or bicyclic heteroaryl containing 1 to 4 heteroatoms independently selected from N, O and S,
wherein aryl and heteroaryl are unsubstituted or substituted with 1 to 7 substituents independently selected from the group consisting of halogen, OH, CN, C₁₋₆-alkyl, O—C₁₋₆-alkyl, O(O)OR^{a}, OC(O)R^{a}, S(O)—C₁₋₆-alkyl, S(O)₂—C₁₋₆-alkyl, N(R^{a})₂, C(O)N(R^{a})₂, NR^{a}C(O)—C₁₋₆-alkyl, S(O)₂N(R^{a})₂, NR^{a}S(O)₂—C₁₋₆-alkyl and C₃₋₆-cycloalkyl, wherein the alkyl and cycloalkyl are unsubstituted or substituted with 1 to 3 substituents independently selected from the group consisting of halogen, C₁₋₃-alkyl, halo-C₁₋₃-alkyl, OH, CN and oxo, or
wherein two substituents on the aryl or heteroaryl group together with the atoms they are attached to may form a 5- to 7-membered saturated or partially unsaturated carbocyclic ring or heterocyclic ring containing 1 to 3 heteroatoms independently selected from O, N and S,
wherein the carbocyclic or heterocyclic ring is unsubstituted or substituted with 1 to 5 substituents independently selected from the group consisting of halogen, C₁₋₆-alkyl and halo-C₁₋₆-alkyl;
B is selected from 6- to 10-membered mono- or bicyclic aryl and 5- to 10-membered mono- or bicyclic heteroaryl containing 1 to 4 heteroatoms independently selected from N, O and S,
wherein aryl and heteroaryl are unsubstituted or substituted with 1 to 7 substituents independently selected from the group consisting of halogen, OH, CN, C₁₋₆-alkyl, O—C₁₋₆-alkyl, C(O)OR^{a}, OC(O)R^{a}, S(O)—C₁₋₆-alkyl, S(O)₂—C₁₋₆-alkyl, N(R^{a})₂, C(O)N(R^{a})₂, NR^{a}C(O)—C₁₋₆-alkyl, S(O)₂N(R^{a})₂, NR^{a}S(O)₂—C₁₋₆-alkyl and C₃₋₆-cycloalkyl,
wherein the alkyl and cycloalkyl are unsubstituted or substituted with 1 to 3 substituents independently selected from the group consisting of halogen, C₁₋₃-alkyl, halo-C₁₋₃-alkyl, OH, CN and oxo, or
wherein two substituents on the aryl or heteroaryl group together with the atoms they are attached to may form a 5- to 7-membered saturated or partially unsaturated carbocyclic ring or heterocyclic ring containing 1 to 3 heteroatoms independently selected from O, N and S,
wherein the carbocyclic or heterocyclic ring is unsubstituted or substituted with 1 to 5 substituents independently selected from the group consisting of halogen, C₁₋₆-alkyl and halo-C₁₋₆-alkyl;
R¹, R², R³ and R⁴ are each independently selected from hydrogen, halogen, C₁₋₄-alkyl halo-C₁₋₃-alkyl, OH, O—C₁₋₃-alkyl, and CN;
R^{a} is independently hydrogen or C₁₋₆-alkyl, and
R^{b} is independently hydrogen or C₁₋₆-alkyl.

The invention further relates to a compound according to the following Formula (I), an enantiomer, diastereomer, tautomer, solvate, prodrug or pharmaceutical acceptable salt thereof

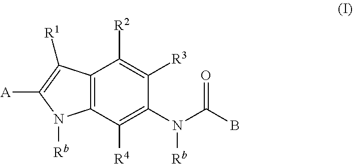

wherein
A is selected from 6- to 10-membered mono- or bicyclic aryl and 5- to 10-membered mono- or bicyclic heteroaryl containing 1 to 4 heteroatoms independently selected from N, O and S,
wherein aryl and heteroaryl are unsubstituted or substituted with 1 to 7 substituents independently selected from the group consisting of halogen, OH, CN, C₁₋₆-alkyl, O—C₁₋₆-alkyl, O(O)OR^{a}, OC(O)R^{a}, S(O)—C₁₋₆-alkyl, S(O)₂—C₁₋₆-alkyl, N(R^{a})₂, C(O)N(R^{a})₂, NR^{a}C(O)—C₁₋₆-alkyl, S(O)₂N(R^{a})₂, NR^{a}S(O)₂—C₁₋₆-alkyl and C₃₋₆-cycloalkyl,
wherein the alkyl and cycloalkyl are unsubstituted or substituted with 1 to 3 substituents independently selected from the group consisting of halogen, C₁₋₃-alkyl, halo-C₁₋₃-alkyl, OH, CN and oxo, or
wherein two substituents on the aryl or heteroaryl group together with the atoms they are attached to may form a 5- to 7-membered saturated or partially unsaturated carbocyclic ring or heterocyclic ring containing 1 to 3 heteroatoms independently selected from O, N and S,
wherein the carbocyclic or heterocyclic ring is unsubstituted or substituted with 1 to 5 substituents independently selected from the group consisting of halogen, C₁₋₆-alkyl and halo-C₁₋₆-alkyl;
B is selected from 6- to 10-membered mono- or bicyclic aryl and 5- to 10-membered mono- or bicyclic heteroaryl containing 1 to 4 heteroatoms independently selected from N, O and S,
wherein aryl and heteroaryl are unsubstituted or substituted with 1 to 7 substituents independently selected from the group consisting of halogen, OH, CN, C₁₋₆-alkyl, O—C₁₋₆-alkyl, O(O)OR^{a}, OC(O)R^{a}, S(O)—C₁₋₆-alkyl, S(O)₂—C₁₋₆-alkyl, N(R^{a})₂, C(O)N(R^{a})₂, NR^{a}C(O)—C₁₋₆-alkyl, S(O)₂N(R^{a})₂, NR^{a}S(O)₂—C₁₋₆-alkyl and C₃₋₆-cycloalkyl,
wherein the alkyl and cycloalkyl are unsubstituted or substituted with 1 to 3 substituents independently selected from the group consisting of halogen, C₁₋₃-alkyl, halo-C₁₋₃-alkyl, OH, CN and oxo, or
wherein two substituents on the aryl or heteroaryl group together with the atoms they are attached to may form a 5- to 7-membered saturated or partially unsaturated carbocyclic ring or heterocyclic ring containing 1 to 3 heteroatoms independently selected from O, N and S,
wherein the carbocyclic or heterocyclic ring is unsubstituted or substituted with 1 to 5 substituents independently selected from the group consisting of halogen, C₁₋₆-alkyl and halo-C₁₋₆-alkyl;
R¹, R², R³ and R⁴ are each independently selected from hydrogen, halogen, C₁₋₄-alkyl halo-C₁₋₃-alkyl, OH, O—C₁₋₃-alkyl, and CN;
R^{a} is independently hydrogen or C₁₋₆-alkyl, and
R^{b} is independently hydrogen or C₁₋₆-alkyl;
with the proviso that the compound is not methyl 4-(6-(4-(dimethylamino)benzamido)-1H-indol-2-yl)benzoate.

In a preferred embodiment in combination with any of the above or below embodiments, R^{b} in the compound according to Formula (I) is hydrogen.

In a preferred embodiment in combination with any of the above or below embodiments, A in the compound according to Formula (I) is selected from 6- to 10-membered mono- or bicyclic aryl and 5- to 10-membered mono- or bicyclic heteroaryl containing 1 to 4 heteroatoms independently selected from N, O and S,

wherein aryl and heteroaryl are unsubstituted or substituted with 1 to 7 substituents independently selected from the group consisting of halogen, OH, CN, C₁₋₆-alkyl, O—C₁₋₆-alkyl, C(O)OR^{a}, OC(O)R^{a}, S(O)—C₁₋₆-alkyl, S(O)₂—C₁₋₆-alkyl, N(R^{a})₂, C(O)N(R^{a})₂, NR^{a}C(O)—C₁₋₆-alkyl, S(O)₂N(R^{a})₂, NR^{a}S(O)₂—C₁₋₆-alkyl and C₃₋₆-cycloalkyl,
wherein the alkyl and cycloalkyl are unsubstituted or substituted with 1 to 3 substituents independently selected from the group consisting of halogen, C₁₋₃-alkyl, halo-C₁₋₃-alkyl, OH, ON and oxo, or
wherein two substituents on the aryl or heteroaryl group together with the atoms they are attached to may form a 5- to 7-membered saturated or partially unsaturated carbocyclic ring or heterocyclic ring containing 1 to 3 heteroatoms independently selected from O, N and S,
wherein the carbocyclic or heterocyclic ring is unsubstituted or substituted with 1 to 5 substituents independently selected from the group consisting of halogen, C₁₋₆-alkyl and halo-C₁₋₆-alkyl;
with the proviso that A is not an unsubstituted or substituted pyrazole ring.

In a preferred embodiment in combination with any of the above or below embodiments, A in the compound according to Formula (I) is unsubstituted or substituted with 1 to 7 substituents independently selected from the group consisting of halogen, OH, CN, C₁₋₆-alkyl, O(O)OR^{a}, OC(O)R^{a}, S(O)—C₁₋₆-alkyl, S(O)₂—C₁₋₆-alkyl, S(O)₂N(R^{a})₂, NR^{a}S(O)₂—C₁₋₆-alkyl and C₃₋₆-cycloalkyl,

wherein the alkyl and cycloalkyl are unsubstituted or substituted with 1 to 3 substituents independently selected from the group consisting of halogen, C₁₋₃-alkyl, halo-C₁₋₃-alkyl, OH, CN and oxo; and
R^{a} is hydrogen or C₁₋₆-alkyl.

In a further preferred embodiment in combination with any of the above or below embodiments, A in the compound according to Formula (I) is substituted with 1 to 5 substituents independently selected from halogen, C₁₋₆-alkyl, C₁₋₆-haloalkyl and C₃₋₆-cycloalkyl

wherein cycloalkyl is unsubstituted or substituted with C₁₋₃-alkyl.

In a preferred embodiment in combination with any of the above or below embodiments, A in the compound of Formula (I) is

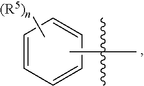

wherein
R⁵ is independently halogen, OH, CN, C₁₋₆-alkyl, O(O)OR^{a}, OC(O)R^{a}, S(O)—C₁₋₆-alkyl, S(O)₂—C₁₋₆-alkyl, S(O)₂N(R^{a})₂, NR^{a}S(O)₂—C₁₋₆-alkyl or C₃₋₆-cycloalkyl,
wherein the alkyl and cycloalkyl are unsubstituted or substituted with 1 to 3 substituents independently selected from the group consisting of halogen, C₁₋₃-alkyl, halo-C₁₋₃-alkyl, OH, CN and oxo;
R^{a} is independently hydrogen or C₁₋₆-alkyl; and
n is 0 to 5.

In a preferred embodiment in combination with any of the above or below embodiments, n in the above formula is 1 to 5 and R⁵ is independently selected from halogen, C₁₋₆-alkyl, C₁₋₆-haloalkyl, and C₃₋₆-cycloalkyl which is unsubstituted or substituted with C₁₋₃-alkyl.

In a preferred embodiment in combination with any of the above or below embodiments, A in the compound of Formula (I) is

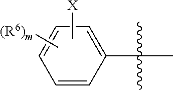

wherein
X is halogen, C₁₋₆-alkyl, C₁₋₆-haloalkyl, or C₃₋₆-cycloalkyl;
R⁶ is halogen; and
m is 0 to 4.

In a preferred embodiment in combination with any of the above or below embodiments, A in the compound of Formula (I) is

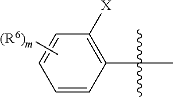

wherein
X is halogen, CH₃, CHF₂ or CF₃;
R⁶ is halogen; and
m is 0 to 4.

In a preferred embodiment in combination with any of the above or below embodiments, B in the compound of Formula (I) is a 5- or 6-membered heteroaryl containing 1 to 4 heteroatoms independently selected from N, O and S, which is unsubstituted or substituted with 1 to 5 substituents independently selected from the group consisting of halogen, OH, CN, C₁₋₆-alkyl, O—C₁₋₆-alkyl, C(O)OR^{a}, OC(O)R^{a}, S(O)—C₁₋₆-alkyl, S(O)₂—C₁₋₆-alkyl, N(R^{a})₂, C(O)N(R^{a})₂, S(O)₂N(R^{a})₂ and C₃₋₆-cycloalkyl,

wherein the alkyl and cycloalkyl are unsubstituted or substituted with 1 to 3 substituents independently selected from the group consisting of halogen, C₁₋₃-alkyl, halo-C₁₋₃-alkyl, OH, CN and oxo; and
R^{a} is hydrogen or C₁₋₆-alkyl.

In a preferred embodiment in combination with any of the above or below embodiments, B in the compound of Formula (I) is unsubstituted or substituted with 1 or 2 substituents independently selected from the group consisting of C₁₋₆-alkyl, C₁₋₆-haloalkyl and C₃₋₆-cycloalkyl.

In a preferred embodiment in combination with any of the above or below embodiments, B in the compound of formula (I) is represented by

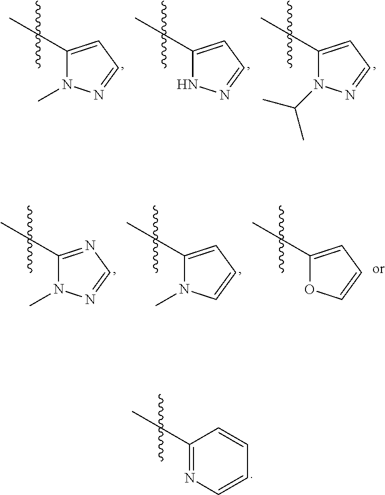

In a more preferred embodiment in combination with any of the above or below embodiments, B in the compound of Formula (I) is represented by

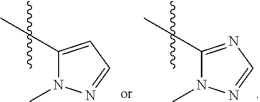

In an even more preferred embodiment in combination with any of the above or below embodiments, B in the compound of Formula (I) is represented by

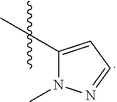 [SMILES: *c1ccnn1C]

In an equally even more preferred embodiment in combination with any of the above or below embodiments, B in the compound of Formula (I) is represented by

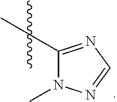 [SMILES: *c1ncnn1C]

In a preferred embodiment in combination with any of the above or below embodiments, B in the compound of Formula (I) is represented by

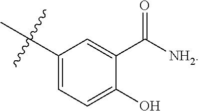 [SMILES: *c1ccc(O)c(C(N)=O)c1]

In a preferred embodiment in combination with any of the above or below embodiments, each of R¹, R², R³ and R⁴ in the compound according to Formula (I) are hydrogen.

In a preferred embodiment in combination with any of the above or below embodiments, the compound of Formula (I) is

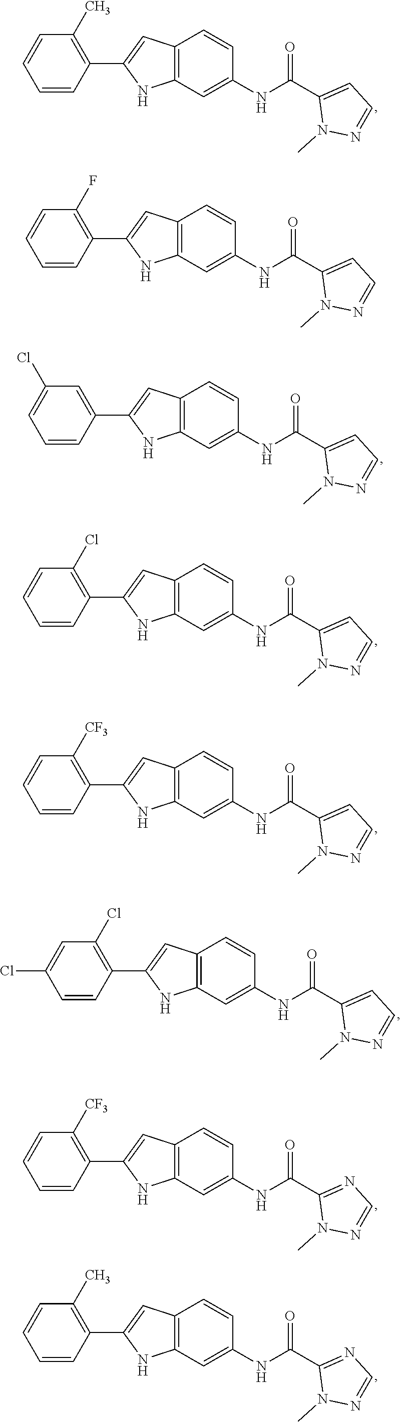

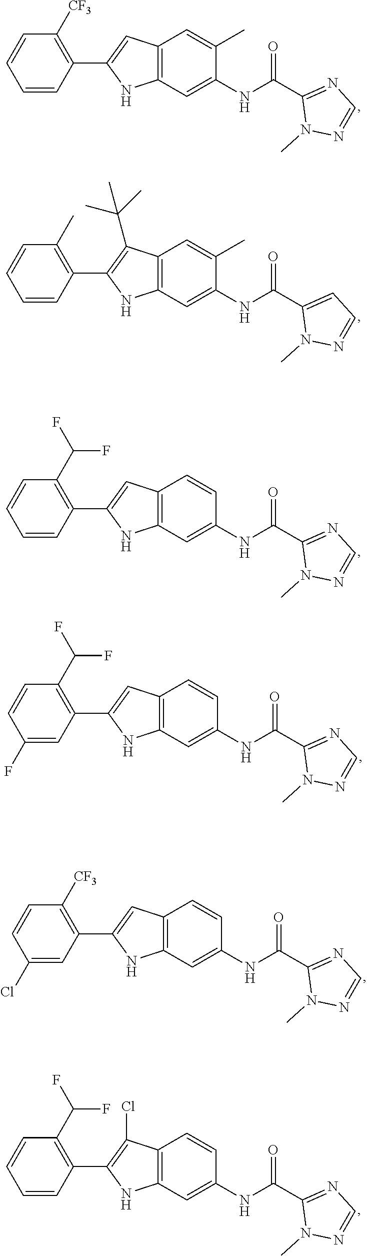 [SMILES: Cc1cc2cc(-c3ccccc3C(F)(F)F)[nH]c2cc1NC(=O)c1ncnn1C ; Cc1cc2c(C(C)(C)C)c(-c3ccccc3C)[nH]c2cc1NC(=O)c1ccnn1C ; Cn1ncnc1C(=O)Nc1ccc2cc(-c3ccccc3C(F)F)[nH]c2c1 ; Cn1ncnc1C(=O)Nc1ccc2cc(-c3cc(F)ccc3C(F)F)[nH]c2c1 ; Cn1ncnc1C(=O)Nc1ccc2cc(-c3cc(Cl)ccc3C(F)(F)F)[nH]c2c1 ; Cn1ncnc1C(=O)Nc1ccc2c(Cl)c(-c3ccccc3C(F)F)[nH]c2c1]

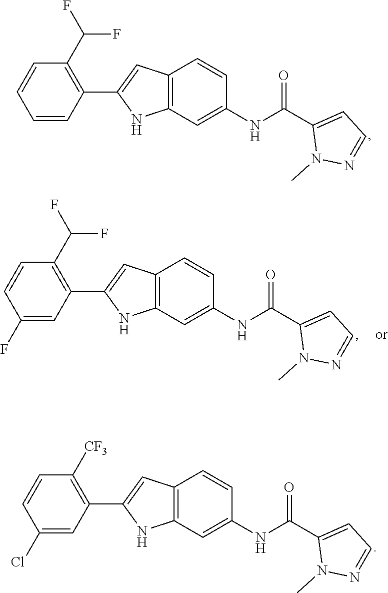 [SMILES: Cn1nccc1C(=O)Nc1ccc2cc(-c3ccccc3C(F)F)[nH]c2c1 ; Cn1nccc1C(=O)Nc1ccc2cc(-c3cc(F)ccc3C(F)F)[nH]c2c1 ; Cn1nccc1C(=O)Nc1ccc2cc(-c3cc(Cl)ccc3C(F)(F)F)[nH]c2c1]

In a further embodiment, the present invention is directed to a pharmaceutical composition comprising a compound according to Formula (I) and a physiologically acceptable excipient.

In a further embodiment, the present invention is directed to a compound according to Formula (I) for use as a medicament.

In yet another embodiment, the present invention is directed to a compound according to Formula (I) or a pharmaceutical composition containing same and a physiologically acceptable excipient for use in the prophylaxis and/or treatment of a disease or condition mediated by aryl hydrocarbon receptor (AhR).

In a preferred embodiment, the disease or condition mediated by aryl hydrocarbon receptor (AhR) is cancer.

In a further preferred embodiment, the compound according to Formula (I) is administered with one or more therapeutic agents for cancer selected from the group consisting of PD-1 agent, PD-L1 agent, CTLA-4 agent, IDO1 inhibitor, chemotherapeutic agent, anticancer vaccine, and cytokine therapy, or wherein the compound is administered under irradiation therapy.

The compounds of the present invention share a common chemical structure according to Formula (I) in claim 1.

In a preferred embodiment in combination with any of the above and below embodiments, A in the compound according to Formula (I) is phenyl or naphthyl which are unsubstituted or substituted with 1 to 7 substituents independently selected from the group consisting of halogen, OH, CN, C₁₋₆-alkyl, C(O)OR^{a}, OC(O)R^{a}, S(O)—C₁₋₆-alkyl, S(O)₂—C₁₋₆-alkyl, S(O)₂N(R^{a})₂, NR^{a}S(O)₂—C₁₋₆-alkyl and C₃₋₆-cycloalkyl,

wherein the alkyl and cycloalkyl are unsubstituted or substituted with 1 to 3 substituents independently selected from the group consisting of halogen, C₁₋₃-alkyl, halo-C₁₋₃-alkyl, OH, CN and oxo, or
wherein two substituents on the phenyl or naphthyl group together with the atoms they are attached to may form a 5- to 7-membered saturated or partially unsaturated carbocyclic ring or heterocyclic ring containing 1 to 3 heteroatoms independently selected from O, N and S,
wherein the carbocyclic or heterocyclic ring is unsubstituted or substituted with 1 to 5 substituents independently selected from the group consisting of halogen, C₁₋₆-alkyl and halo-C₁₋₆-alkyl, and
R^{a} is hydrogen or C₁₋₆-alkyl, more preferably hydrogen.

In a further preferred embodiment in combination with any of the above and below embodiments, A in the compound according to Formula (I) is 5- to 10-membered mono-or bicyclic heteroaryl containing 1 to 4 heteroatoms independently selected from N, O and S, wherein aryl and heteroaryl are unsubstituted or substituted with 1 to 7 substituents independently selected from the group consisting of halogen, OH, CN, C₁₋₆-alkyl, O—C₁₋₆-alkyl, O(O)OR^{a}, OC(O)R^{a}, S(O)—C₁₋₆-alkyl, S(O)₂—C₁₋₆-alkyl, N(R^{a})₂, C(O)N(R^{a})₂, NR^{a}C(O)—C₁₋₆-alkyl, S(O)₂N(R^{a})₂, NR^{a}S(O)₂—C₁₋₆-alkyl and C₃₋₆-cycloalkyl,

wherein the alkyl and cycloalkyl are unsubstituted or substituted with 1 to 3 substituents independently selected from the group consisting of halogen, C₁₋₃-alkyl, halo-C₁₋₃-alkyl, OH, CN and oxo, or
wherein two substituents on the aryl or heteroaryl group together with the atoms they are attached to may form a 5- to 7-membered saturated or partially unsaturated carbocyclic ring or heterocyclic ring containing 1 to 3 heteroatoms independently selected from O, N and S,
wherein the carbocyclic or heterocyclic ring is unsubstituted or substituted with 1 to 5 substituents independently selected from the group consisting of halogen, C₁₋₆-alkyl and halo-C₁₋₆-alkyl,
and R^{a} is hydrogen or C₁₋₆-alkyl, more preferably hydrogen.

In a more preferred embodiment in combination with any of the above and below embodiments, A is a 5- or 6-membered monocyclic heteroaryl containing 1 to 4 heteroatoms, more preferably 1 to 3 heteroatoms, independently selected from N, O and S which heteroaryl is unsubstituted or substituted as above. More preferably, the heteroatoms are independently selected from N and O.

In an equally more preferred embodiment in combination with any of the above and below embodiments, A in the compound according to Formula (I) is a 9- to 10-membered bicyclic heteroaryl containing 1 to 4 heteroatoms, more preferably 1 to 3 heteroatoms, independently selected from N, O and S, which heteroaryl is unsubstituted or substituted as above. More preferably, the heteroatoms are independently selected from N and O.

In a preferred embodiment in combination with any of the above and below embodiments, A in the compound according to Formula (I) is

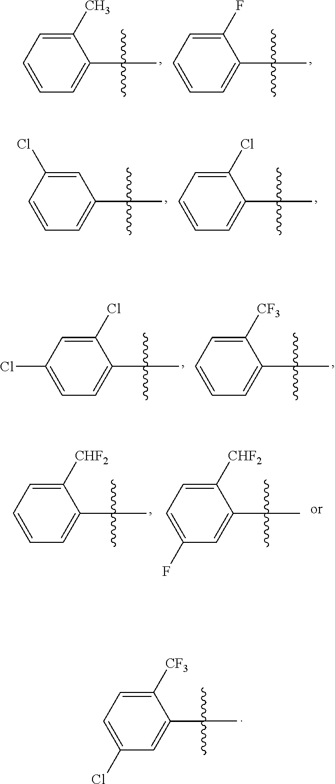

In a more preferred embodiment in combination with any of the above and below embodiments, A in the compound according to Formula (I) is

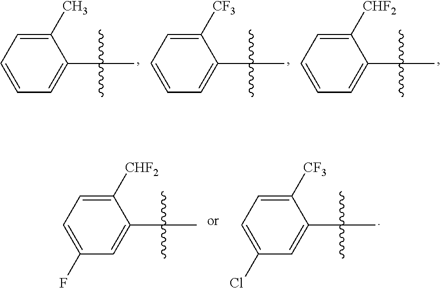

In a most preferred embodiment in combination with any of the above and below embodiments, A in the compound according to Formula (I) is

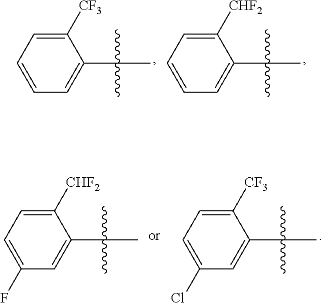 [SMILES: *c1ccccc1C(F)(F)F ; *c1ccccc1C(F)F ; *c1cc(F)ccc1C(F)F ; *c1cc(Cl)ccc1C(F)(F)F]

In a preferred embodiment in combination with any of the above and below embodiments, B in the compound according to Formula (I) is phenyl or naphthyl

wherein phenyl and naphthyl are unsubstituted or substituted with 1 to 7 substituents independently selected from the group consisting of halogen, OH, CN, C₁₋₆-alkyl, O—C₁₋₆-alkyl, C(O)OR^{a}, OC(O)R^{a}, S(O)—C₁₋₆-alkyl, S(O)₂—C₁₋₆-alkyl, N(R^{a})₂, C(O)N(R^{a})₂, NR^{a}C(O)—C₁₋₆-alkyl, S(O)₂N(R^{a})₂, NR^{a}S(O)₂—C₁₋₆-alkyl and C₃₋₆-cycloalkyl,
wherein the alkyl and cycloalkyl are unsubstituted or substituted with 1 to 3 substituents independently selected from the group consisting of halogen, C₁₋₃-alkyl, halo-C₁₋₃-alkyl, OH, CN and oxo, or
wherein two substituents on the aryl or heteroaryl group together with the atoms they are attached to may form a 5- to 7-membered saturated or partially unsaturated carbocyclic ring or heterocyclic ring containing 1 to 3 heteroatoms independently selected from O, N and S,
wherein the carbocyclic or heterocyclic ring is unsubstituted or substituted with 1 to 5 substituents independently selected from the group consisting of halogen, C₁₋₆-alkyl and halo-C₁₋₆-alkyl, and
R^{a} is hydrogen or C₁₋₆-alkyl, more preferably hydrogen.

In a further preferred embodiment in combination with any of the above and below embodiments, B in the compound according Formula (I) is phenyl which is unsubstituted or substituted with 1 to 5 substituents independently selected from the group consisting of halogen, OH, CN, C₁₋₆-alkyl, O—C₁₋₆-alkyl, C(O)OR^{a}, OC(O)R^{a}, S(O)—C₁₋₆-alkyl, S(O)₂—C₁₋₆-alkyl, N(R^{a})₂, C(O)N(R^{a})₂, S(O)₂N(R^{a})₂ and C₃₋₆-cycloalkyl,

wherein the alkyl and cycloalkyl are unsubstituted or substituted with 1 to 3 substituents independently selected from the group consisting of halogen, C₁₋₃-alkyl, halo-C₁₋₃-alkyl, OH, CN and oxo; and
R^{a} is hydrogen or C₁₋₆-alkyl, more preferably hydrogen.

In an equally preferred embodiment in combination with any of the above and below embodiments, B is a 9- or 10-membered bicyclic heteroaryl containing 1 to 4 heteroatoms, more preferably 1 to 3 heteroatoms, independently selected from N, O and S, which heteroaryl is unsubstituted or substituted with 1 to 5 substituents independently selected from the group consisting of halogen, OH, CN, C₁₋₆-alkyl, O—C₁₋₆-alkyl, C(O)OR^{a}, OC(O)R^{a}, S(O)—C₁₋₆-alkyl, S(O)₂—C₁₋₆-alkyl, N(R^{a})₂, C(O)N(R^{a})₂, S(O)₂N(R^{a})₂ and C₃₋₆-cycloalkyl,

wherein the alkyl and cycloalkyl are unsubstituted or substituted with 1 to 3 substituents independently selected from the group consisting of halogen, C₁₋₃-alkyl, halo-C₁₋₃-alkyl, OH, ON and oxo; and
R^{a} is hydrogen or C₁₋₆-alkyl, more preferably hydrogen.

In a further preferred embodiment in combination with any of the above and below embodiments, B is a 5- or 6-membered monocyclic heteroaryl containing 1 to 4 heteroatoms, more preferably 1 to 3 heteroatoms independently selected from N, O and S, more preferably from N and O, which is unsubstituted or substituted as above.

In a preferred embodiment in combination with any of the above or below embodiments, B in the compound according to Formula (I) is a 5-membered heteroaryl containing 1 to 3 heteroatoms independently selected from N, O and S, more preferably 2 or 3 nitrogen atoms, wherein the 5-membered heteroaryl is unsubstituted or substituted with 1 or 2 substituents independently selected from C₁₋₆-alkyl, halo-C₁₋₆-alkyl and C₃₋₆-cycloalkyl.

In a preferred embodiment in combination with any of the above and below embodiments, R¹, R², R³ and R⁴ are independently selected from hydrogen, halogen, C₁₋₄-alkyl, halo-C₁₋₃-alkyl, OH and CN. More preferably, one of R¹, R², R³ and R⁴ is halogen, C₁₋₄-alkyl, halo-C₁₋₃-alkyl, OH and CN and the other three are hydrogen. Even more preferred, one of R¹, R², R³ and R⁴ is C₁₋₄-alkyl and the other three are hydrogen. Most preferably, each of R¹, R², R³ and R⁴ is hydrogen.

In a preferred embodiment in combination with any of the above and below embodiments, the compound according to Formula (I) is selected from

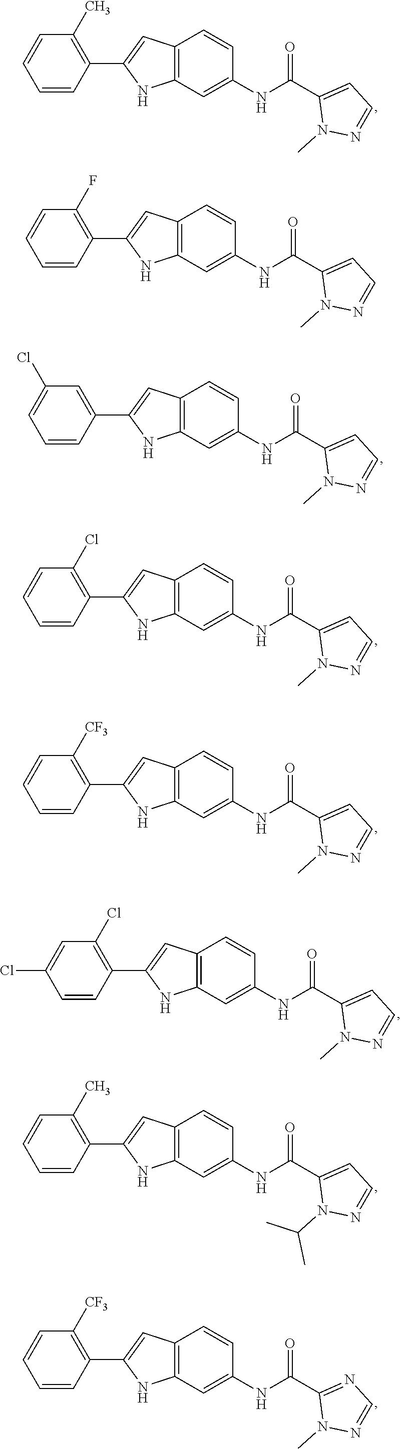

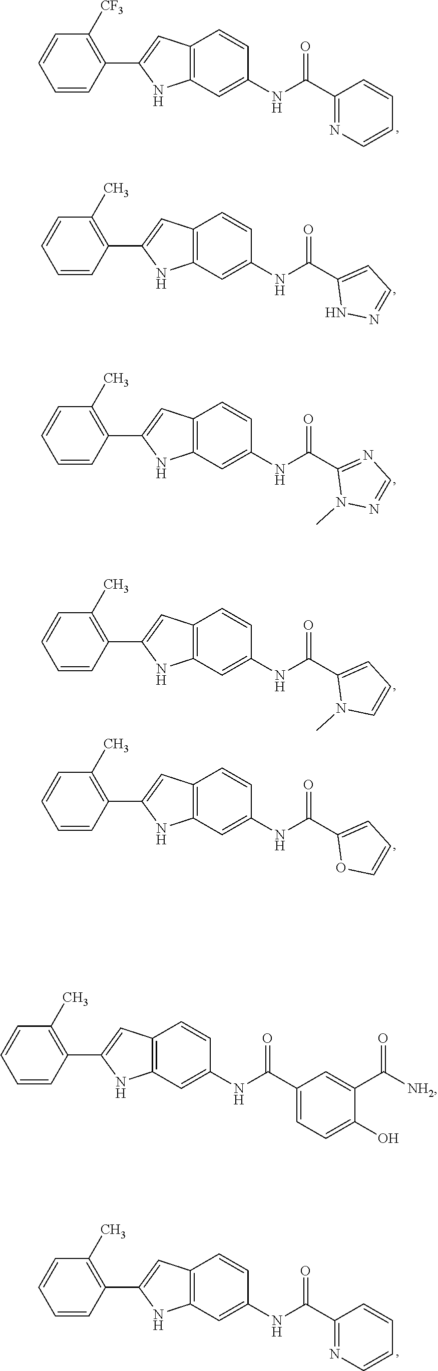

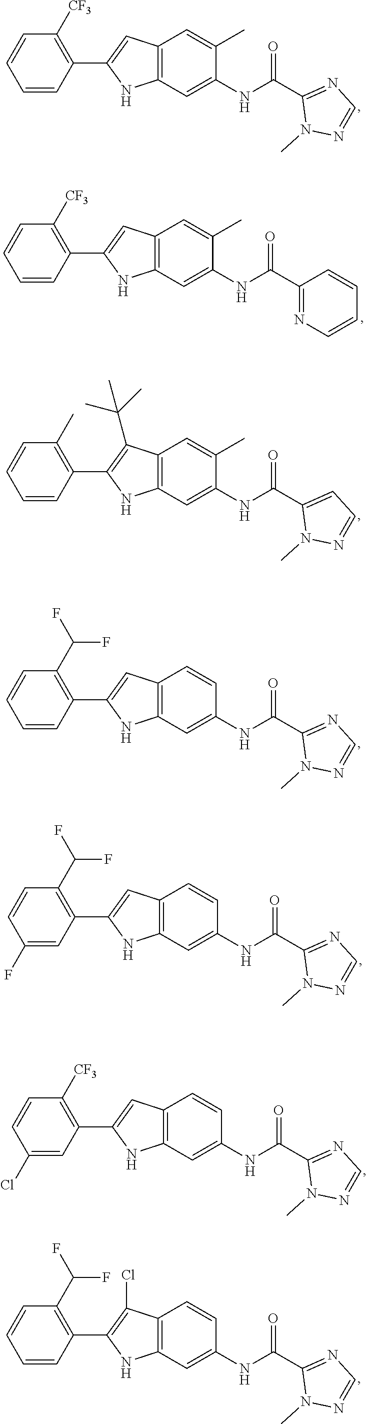

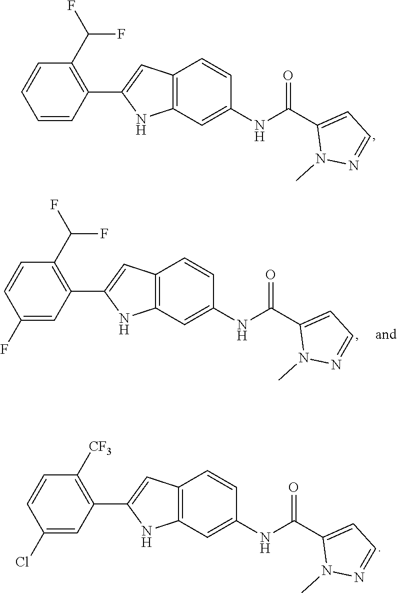 [SMILES: Cn1nccc1C(=O)Nc1ccc2cc(-c3ccccc3C(F)F)[nH]c2c1 ; Cn1nccc1C(=O)Nc1ccc2cc(-c3cc(F)ccc3C(F)F)[nH]c2c1 ; Cn1nccc1C(=O)Nc1ccc2cc(-c3cc(Cl)ccc3C(F)(F)F)[nH]c2c1]

In a more preferred embodiment in combination with any of the above and below embodiments, the compound according to Formula (I) is selected from

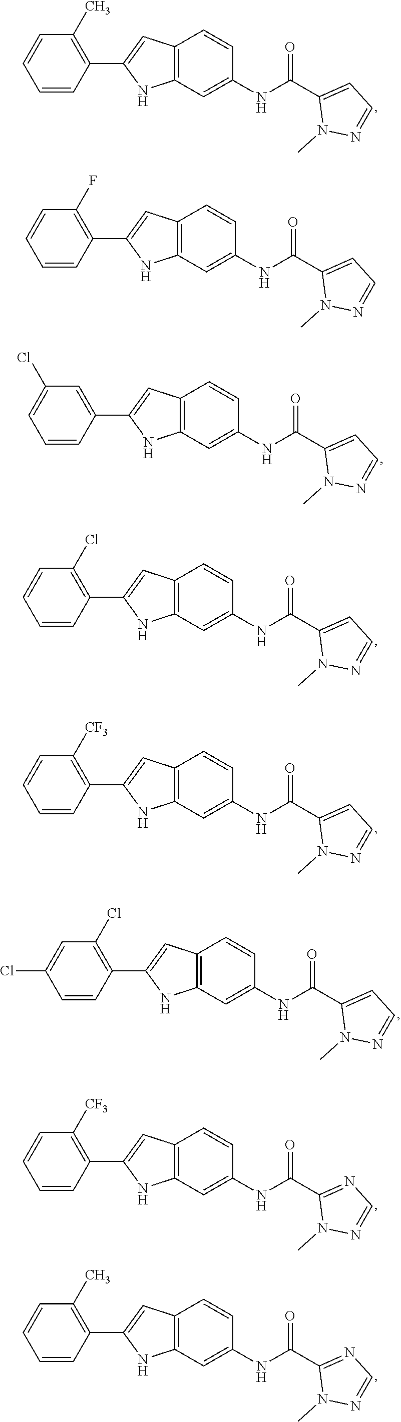

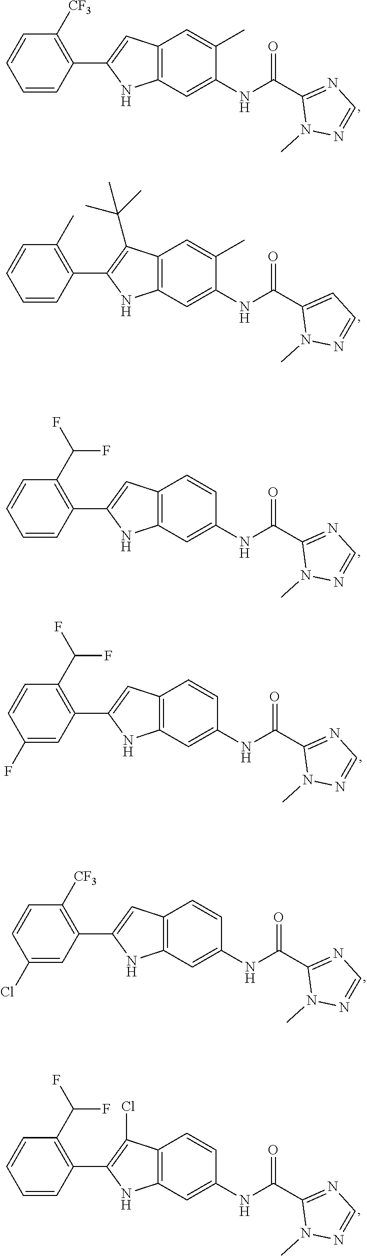 [SMILES: Cc1cc2cc(-c3ccccc3C(F)(F)F)[nH]c2cc1NC(=O)c1ncnn1C ; Cc1cc2c(C(C)(C)C)c(-c3ccccc3C)[nH]c2cc1NC(=O)c1ccnn1C ; Cn1ncnc1C(=O)Nc1ccc2cc(-c3ccccc3C(F)F)[nH]c2c1 ; Cn1ncnc1C(=O)Nc1ccc2cc(-c3cc(F)ccc3C(F)F)[nH]c2c1 ; Cn1ncnc1C(=O)Nc1ccc2cc(-c3cc(Cl)ccc3C(F)(F)F)[nH]c2c1 ; Cn1ncnc1C(=O)Nc1ccc2c(Cl)c(-c3ccccc3C(F)F)[nH]c2c1]

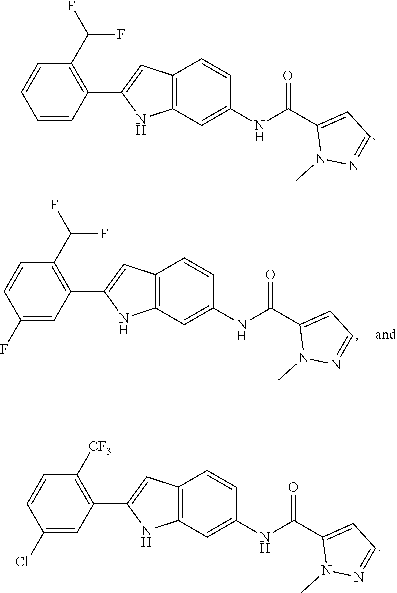 [SMILES: Cn1nccc1C(=O)Nc1ccc2cc(-c3ccccc3C(F)F)[nH]c2c1 ; Cn1nccc1C(=O)Nc1ccc2cc(-c3cc(F)ccc3C(F)F)[nH]c2c1 ; Cn1nccc1C(=O)Nc1ccc2cc(-c3cc(Cl)ccc3C(F)(F)F)[nH]c2c1]

In a most preferred embodiment in combination with any of the above and below embodiments, the compound according to Formula (I) is selected from

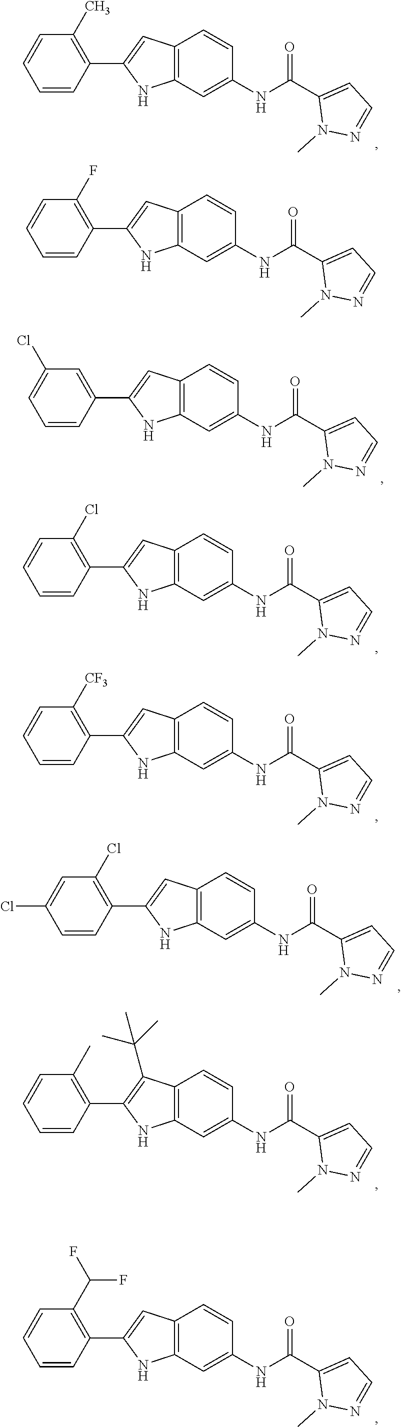

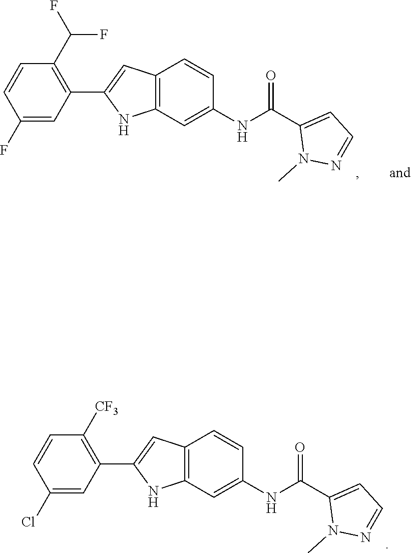 [SMILES: Cn1nccc1C(=O)Nc1ccc2cc(-c3cc(F)ccc3C(F)F)[nH]c2c1 ; Cn1nccc1C(=O)Nc1ccc2cc(-c3cc(Cl)ccc3C(F)(F)F)[nH]c2c1]

In an equally most preferred embodiment in combination with any of the above and below embodiments, the compound according to Formula (I) is selected from

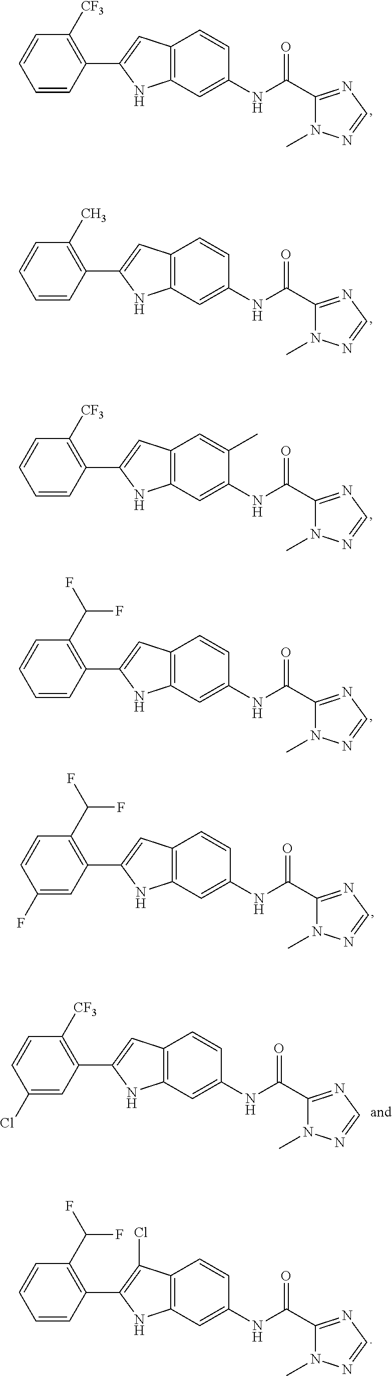

In the context of the present invention “C₁₋₆-alkyl” means a saturated alkyl chain having 1 to 6 carbon atoms which may be straight chained or branched. Examples thereof include methyl, ethyl, propyl, isopropyl, n-butyl, isobutyl, tert-butyl, n-pentyl, isopentyl, neopentyl, and hexyl.

The term “O—C₁₋₆-alkyl” means that the alkyl chain is connected via an oxygen atom with the remainder of the molecule.

The term “halo-C₁₋₁₀-alkyl” means that one or more hydrogen atoms in the alkyl chain are replaced by a halogen. A preferred example thereof is CF₃.

A C₃₋₆-cycloalkyl group means a saturated or partially unsaturated mono- or bicyclic ring system comprising 3 to 6 carbon atoms. Examples include cyclopropyl, cyclobutyl, cyclopentyl, and cyclohexyl.

A 5-10-membered mono- or bicyclic heteroaromatic ring system (within the application also referred to as heteroaryl) containing up to 4 heteroatoms means a monocyclic heteroaromatic ring such as pyrrolyl, imidazolyl, furanyl, thiophenyl, pyridinyl, pyrimidinyl, pyrazinyl, pyrazolyl, oxazolyl, isoxazolyl, triazolyl, oxadiazolyl and thiadiazolyl. It further means a bicyclic ring system wherein the heteroatom(s) may be present in one or both rings including the bridgehead atoms. Examples thereof include quinolinyl, isoquinolinyl, quinoxalinyl, benzimidazolyl, benzisoxazolyl, benzodioxanyl, benzofuranyl, benzoxazolyl, indolyl, indolizinyl, pyrazolo[1,5-a]pyrimidinyl and dibenzo[b,d]furanyl. The nitrogen or sulphur atom of the heteroaryl system may also be optionally oxidized to the corresponding N-oxide, S-oxide or S,S-dioxide. If not stated otherwise, the heteroaryl system can be connected via a carbon or nitrogen atom.

Examples for N-linked heterocycles are

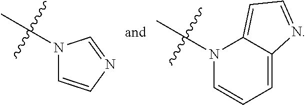 [SMILES: *n1ccnc1 ; *n1cccc2nccc1-2]

Moreover, where not explicitly defined, heteroaryl contains 1 to 4 heteroatoms independently selected from the group consisting of N, O and S.

A 6-10-membered mono- or bicyclic aromatic ring system (within the application also referred to as aryl) means an aromatic carbon cycle such as phenyl or naphthyl.

The term “halogen” comprises the specific halogen atoms fluorine, bromine, chlorine and iodine.

Any formula or structure given herein, is also intended to represent unlabeled forms as well as isotopically labeled forms of the compounds. Isotopically labeled compounds have structures depicted by the formulas given herein except that one or more atoms are replaced by an atom having a selected atomic mass or mass number. Examples of isotopes that can be incorporated into compounds of the disclosure include isotopes of hydrogen, carbon, nitrogen, oxygen, fluorine and chlorine, such as, but not limited to ²H (deuterium, D), ³H (tritium), ¹¹C, ¹³C, ¹⁴C, ¹⁵N, ¹⁸F, ³⁵S, ³⁶Cl and ¹²⁵I. Various isotopically labeled compounds of the present disclosure, for example those into which radioactive isotopes such as ³H, ¹³C and ¹⁴C are incorporated. Such isotopically labelled compounds may be useful in metabolic studies, reaction kinetic studies, detection or imaging techniques, such as positron emission tomography (PET) or single-photon emission computed tomography (SPECT) including drug or substrate tissue distribution assays or in radioactive treatment of patients. Isotopically labeled compounds of this disclosure and prodrugs thereof can generally be prepared by carrying out the procedures disclosed in the schemes or in the examples and preparations described below by substituting a readily available isotopically labeled reagent for a non-isotopically labeled reagent.

The disclosure also includes “deuterated analogs” of compounds of Formula (I) in which from 1 to n hydrogens attached to a carbon atom is/are replaced by deuterium, in which n is the number of hydrogens in the molecule. Such compounds may exhibit increased resistance to metabolism and thus be useful for increasing the half-life of any compound of Formula (I) when administered to a mammal, e.g. a human. See, for example, Foster in Trends Pharmacol. Sci. 1984:5; 524. Such compounds are synthesized by means well known in the art, for example by employing starting materials in which one or more hydrogens have been replaced by deuterium.

Deuterium labelled or substituted therapeutic compounds of the disclosure may have improved DMPK (drug metabolism and pharmacokinetics) properties, relating to distribution, metabolism and excretion (ADME). Substitution with heavier isotopes such as deuterium may afford certain therapeutic advantages resulting from greater metabolic stability, for example increased in vivo half-life, reduced dosage requirements and/or an improvement in therapeutic index. An ¹⁸F labeled compound may be useful for PET or SPECT studies.

The concentration of such a heavier isotope, specifically deuterium, may be defined by an isotopic enrichment factor. In the compounds of this disclosure any atom not specifically designated as a particular isotope is meant to represent any stable isotope of that atom. Unless otherwise stated, when a position is designated specifically as “H” or “hydrogen”, the position is understood to have hydrogen at its natural abundance isotopic composition. Accordingly, in the compounds of this disclosure any atom specifically designated as a deuterium (D) is meant to represent deuterium.

The compounds of the present invention can be in the form of a prodrug compound. “Prodrug compound” means a derivative that is converted into a compound according to the present invention by a reaction with an enzyme, gastric acid or the like under a physiological condition in the living body, e.g. by oxidation, reduction, hydrolysis or the like, each of which is carried out enzymatically. Examples of the prodrug are compounds, wherein the amino group in a compound of the present invention is acylated, alkylated or phosphorylated to form, e.g., eicosanoylamino, alanylamino, pivaloyloxymethylamino or wherein the hydroxyl group is acylated, alkylated, phosphorylated or converted into the borate, e.g. acetyloxy, palmitoyloxy, pivaloyloxy, succinyloxy, fumaryloxy, alanyloxy or wherein the carboxyl group is esterified or amidated. These compounds can be produced from compounds of the present invention according to well-known methods. Other examples of the prodrug are compounds, wherein the carboxylate in a compound of the present invention is, for example, converted into an alkyl-, aryl-, choline-, amino, acyloxymethylester, linolenoylester.

Metabolites of compounds of the present invention are also within the scope of the present invention.

Where tautomerism, like e.g. keto-enol tautomerism, of compounds of the present invention or their prodrugs may occur, the individual forms, like e.g. the keto and enol form, are each within the scope of the invention as well as their mixtures in any ratio. Same applies for stereoisomers, like e.g. enantiomers, cis/trans isomers, conformers and the like.

If desired, isomers can be separated by methods well known in the art, e.g. by liquid chromatography. Same applies for enantiomers by using e.g. chiral stationary phases. Additionally, enantiomers may be isolated by converting them into diastereomers, i.e. coupling with an enantiomerically pure auxiliary compound, subsequent separation of the resulting diastereomers and cleavage of the auxiliary residue. Alternatively, any enantiomer of a compound of the present invention may be obtained from stereoselective synthesis using optically pure starting materials. Another way to obtain pure enantiomers from racemic mixtures would use enantioselective crystallization with chiral counterions.

The compounds of the present invention can be in the form of a pharmaceutically acceptable salt or a solvate. The term “pharmaceutically acceptable salts” refers to salts prepared from pharmaceutically acceptable non-toxic bases or acids, including inorganic bases or acids and organic bases or acids. In case the compounds of the present invention contain one or more acidic or basic groups, the invention also comprises their corresponding pharmaceutically or toxicologically acceptable salts, in particular their pharmaceutically utilizable salts. Thus, the compounds of the present invention which contain acidic groups can be present on these groups and can be used according to the invention, for example, as alkali metal salts, alkaline earth metal salts or ammonium salts. More precise examples of such salts include sodium salts, potassium salts, calcium salts, magnesium salts or salts with ammonia or organic amines such as, for example, ethylamine, ethanolamine, triethanolamine or amino acids. The compounds of the present invention which contain one or more basic groups, i.e. groups which can be protonated, can be present and can be used according to the invention in the form of their addition salts with inorganic or organic acids. Examples of suitable acids include hydrogen chloride, hydrogen bromide, phosphoric acid, sulfuric acid, nitric acid, methanesulfonic acid, p-toluenesulfonic acid, naphthalenedisulfonic acids, oxalic acid, acetic acid, tartaric acid, lactic acid, salicylic acid, benzoic acid, formic acid, propionic acid, pivalic acid, diethylacetic acid, malonic acid, succinic acid, pimelic acid, fumaric acid, maleic acid, malic acid, sulfaminic acid, phenylpropionic acid, gluconic acid, ascorbic acid, isonicotinic acid, citric acid, adipic acid, and other acids known to the person skilled in the art. If the compounds of the present invention simultaneously contain acidic and basic groups in the molecule, the invention also includes, in addition to the salt forms mentioned, inner salts or betaines (zwitterions). The respective salts can be obtained by customary methods which are known to the person skilled in the art like, for example, by contacting these with an organic or inorganic acid or base in a solvent or dispersant, or by anion exchange or cation exchange with other salts. The present invention also includes all salts of the compounds of the present invention which, owing to low physiological compatibility, are not directly suitable for use in pharmaceuticals but which can be used, for example, as intermediates for chemical reactions or for the preparation of pharmaceutically acceptable salts.

Further the compounds of the present invention may be present in the form of solvates, such as those which include as solvate water, or pharmaceutically acceptable solvates, such as alcohols, in particular ethanol.

Furthermore, the present invention provides pharmaceutical compositions comprising at least one compound of the present invention, or a prodrug compound thereof, or a pharmaceutically acceptable salt or solvate thereof as active ingredient together with a pharmaceutically acceptable carrier.

“Pharmaceutical composition” means one or more active ingredients, and one or more inert ingredients that make up the carrier, as well as any product which results, directly or indirectly, from combination, complexation or aggregation of any two or more of the ingredients, or from dissociation of one or more of the ingredients, or from other types of reactions or interactions of one or more of the ingredients. Accordingly, the pharmaceutical compositions of the present invention encompass any composition made by admixing at least one compound of the present invention and a pharmaceutically acceptable carrier.

The pharmaceutical composition of the present invention may additionally comprise one or more other compounds as active ingredients like a prodrug compound or other nuclear receptor modulators.

In practical use, the compounds used in the present invention can be combined as the active ingredient in intimate admixture with a pharmaceutical carrier according to conventional pharmaceutical compounding techniques. The carrier may take a wide variety of forms depending on the form of preparation desired for administration, e.g., oral or parenteral (including intravenous). In preparing the compositions for oral dosage form, any of the usual pharmaceutical media may be employed, such as, for example, water, glycols, oils, alcohols, flavouring agents, preservatives, colouring agents and the like in the case of oral liquid preparations, such as, for example, suspensions, elixirs and solutions; or carriers such as starches, sugars, microcrystalline cellulose, diluents, granulating agents, lubricants, binders, disintegrating agents and the like in the case of oral solid preparations such as, for example, powders, hard and soft capsules and tablets, with the solid oral preparations being preferred over the liquid preparations.

Because of their ease of administration, tablets and capsules represent the most advantageous oral dosage unit form in which case solid pharmaceutical carriers are obviously employed. If desired, tablets may be coated by standard aqueous or non-aqueous techniques. Such compositions and preparations should contain at least 0.1 percent of active compound. The percentage of active compound in these compositions may, of course, be varied and may conveniently be between about 2 percent to about 60 percent of the weight of the unit. The amount of active compound in such therapeutically useful compositions is such that an effective dosage will be obtained. The active compounds can also be administered intranasally as, for example, liquid drops or spray.

The tablets, pills, capsules, and the like may also contain a binder such as gum tragacanth, acacia, corn starch or gelatin; excipients such as dicalcium phosphate; a disintegrating agent such as corn starch, potato starch, alginic acid; a lubricant such as magnesium stearate; and a sweetening agent such as sucrose, lactose or saccharin. When a dosage unit form is a capsule, it may contain, in addition to materials of the above type, a liquid carrier such as a fatty oil.

Various other materials may be present as coatings or to modify the physical form of the dosage unit. For instance, tablets may be coated with shellac, sugar or both. A syrup or elixir may contain, in addition to the active ingredient, sucrose as a sweetening agent, methyl and propylparabens as preservatives, a dye and a flavouring such as cherry or orange flavour.

The compounds used in the present invention may also be administered parenterally. Solutions or suspensions of these active compounds can be prepared in water suitably mixed with a surfactant such as hydroxy-propylcellulose. Dispersions can also be prepared in glycerol, liquid polyethylene glycols and mixtures thereof in oils. Under ordinary conditions of storage and use, these preparations contain a preservative to prevent the growth of microorganisms.

The pharmaceutical forms suitable for injectable use include sterile aqueous solutions or dispersions and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (e.g., glycerol, propylene glycol and liquid polyethylene glycol), suitable mixtures thereof, and vegetable oils.

Any suitable route of administration may be employed for providing a mammal, especially a human, with an effective dose of a compound of the present invention. For example, oral, rectal, topical, parenteral (including intravenous, intramuscular and subcutaneous), ocular (ophthalmic), pulmonary (nasal or buccal inhalation), nasal, and the like may be employed. Dosage forms include tablets, troches, dispersions, suspensions, solutions, capsules, creams, ointments, aerosols, and the like. Preferably compounds of the present invention are administered orally.

The effective dosage of active ingredient employed may vary depending on the particular compound employed, the mode of administration, the condition being treated and the severity of the condition being treated. Such dosage may be ascertained readily by a person skilled in the art.

When treating or preventing AhR-mediated conditions for which compounds of Formula (I) are indicated, generally satisfactory results are obtained when the compounds are administered at a daily dosage of from about 0.1 mg to about 100 mg per kilogram of mammal body weight, preferably given as a single daily dose or in divided doses two to six times a day, or in sustained release form. For most large mammals, the total daily dosage is from about 1 mg to about 1000 mg, preferably from about 1 mg to about 50 mg. In the case of a 70 kg adult human, the total daily dose will generally be from about 7 mg to about 350 mg. This dosage regimen may be adjusted to provide the optimal therapeutic response.

### Abbreviations

Herein and throughout the application, the following abbreviations may be used.
Ac acetyl
Boc tert-butyloxycarbonyl
br broad
CDI 1,1′-carbonyldiimidazole
d doublet
DAST diethylaminosulfur trifluoride
DCM dichloromethane
dba dibenzylideneacetone
DBU 1,8-diazabicyclo[5.4.0]undec-7-ene
DIBAL-H diisobutylaluminum hydride
DIPEA N,N-diisopropylethylamine
DMAP 4-(dimethylamino)pyridine
DMF N,N-dimethylformamide
DMSO dimethyl sulfoxide
dppf 1,1′-bis(diphenylphosphanyl) ferrocene
EDC 1-ethyl-3-(3-dimethylaminopropyl)carbodiimide
Et ethyl
Et₂O diethyl ether
EtOAc ethyl acetate
HATU O-(7-azabenzotriazol-1-yl)-N,N,N′,N′tetramethyluronium hexafluorophosphate
HPLC high performance liquid chromatography
m multiplet
Me methyl
MCPBA 3-chloroperoxybenzoic acid
Ms methanesulfonyl
NCS N-chlorosuccinimide
PE petroleum ether
prep preparative
rt room temperature
s singlet
t triplet
TEA triethylamine
TFA trifluoroacetic acid
THF tetrahydrofurane

### General Schemes

The compounds of the present invention can be prepared by a combination of methods known in the art including the procedures described in schemes 1 and 2 below. The following reaction schemes are only meant to represent examples of the invention and are in no way meant to be a limit of the invention.

Scheme 1 describes the route of preparation for the compounds of the present invention starting from boronic acids. A substituted or unsubstituted (6-bromo-1-(tert-butoxycarbonyl)-1H-indol-2-yl)boronic acid A-1 is converted by Suzuki coupling with an aryl halide to give intermediate A-2. Buchwald amidation affords the corresponding amide A-3 which is converted into compounds of structure A-5 with for example TFA. Alternatively intermediate A-2 is converted to a Boc-protected aryl amine A-4 which is converted into compounds of structure A-5 via a sequence of Boc-deprotection followed by amide coupling.

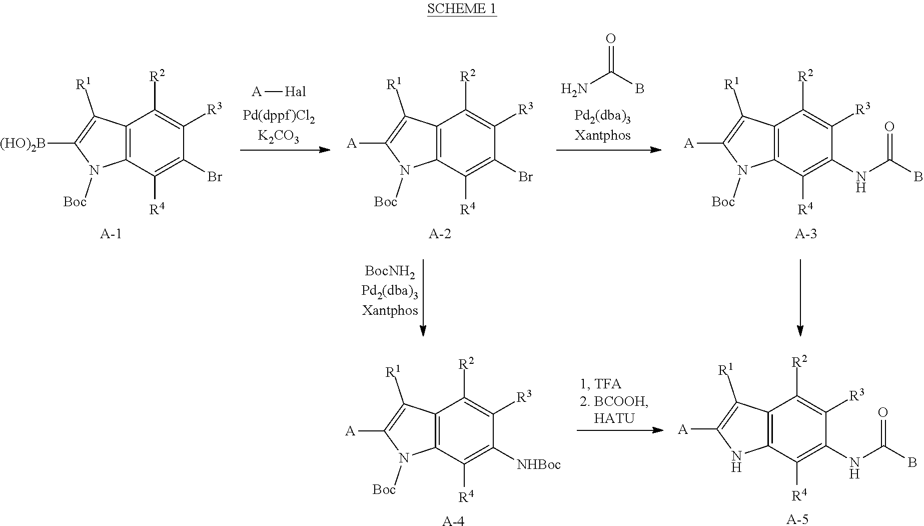

Scheme 2 describes an alternative route of preparation for the compounds of the present invention. A 2-bromo-5-nitroaniline B-1 is converted to N-(2-bromo-5-nitrophenyl)-N-(methylsulfonyl)methanesulfonamide B-2. Treatment of B-2 with NaOH affords the corresponding mono methanesulfonamide B-3 which is converted to indole B-4 via Pd/Cu(I) catalysed coupling/cyclisation reaction with an appropriately substituted alkyne. Boc-protection to intermediate B-5 followed by reduction with Fe/NH₄Cl gives the amino intermediate B6. A sequence of amide coupling with an appropriate carboxylic acid followed by deprotection gives the compounds of structure A-5.

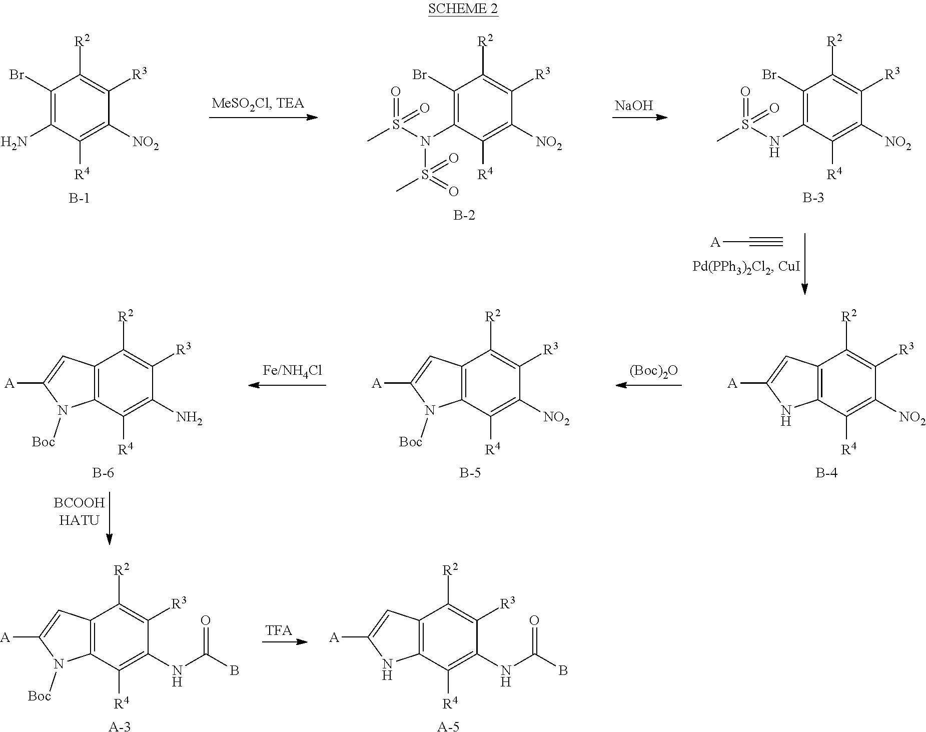

### Intermediate 1: tert-Butyl 6-bromo-2-(o-tolyl)-1H-indole-1-carboxylate (Int 1)

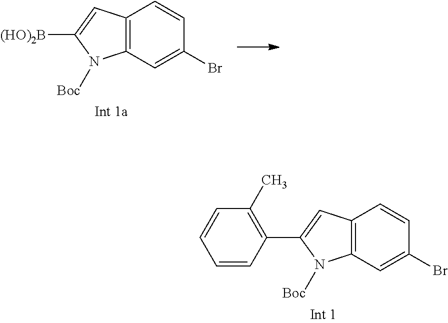 [SMILES: CC(C)(C)OC(=O)n1c(B(O)O)cc2ccc(Br)cc21 ; Cc1ccccc1-c1cc2ccc(Br)cc2n1C(=O)OC(C)(C)C]

A mixture of (6-bromo-1-(tert-butoxycarbonyl)-1H-indol-2-yl)boronic acid (850 mg, 2.5 mmol), 1-iodo-2-methylbenzene (1.6 g, 7.5 mmol), Pd(dppf)Cl₂ (178 mg, 0.25 mmol) and K₂CO₃ (690 mg, 5.0 mmol) in 1,4-dioxane/water (40 mL, 3/1) was stirred at 110° C. under N₂ atmosphere for 2 h. The layers were separated and the organic layer was concentrated to dryness. The residue was purified by column chromatography (PE/EtOAc=97:3) to give the title compound as a white solid.

### Intermediate 1/1: tert-Butyl 6-bromo-2-(2-fluorophenyl)-1H-indole-1-carboxylate (Int 1/1)

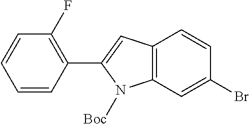 [SMILES: CC(C)(C)OC(=O)n1c(-c2ccccc2F)cc2ccc(Br)cc21]

The title compound was prepared similar as described for Intermediate 1 using 1-fluoro-2-iodobenzene in place of 1-iodo-2-methylbenzene.

### Intermediate 1/2: tert-Butyl 6-bromo-2-(2-chlorophenyl)-1H-indole-1-carboxylate (Int 1/2)

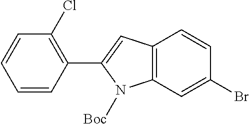 [SMILES: CC(C)(C)OC(=O)n1c(-c2ccccc2Cl)cc2ccc(Br)cc21]

The title compound was prepared similar as described for Intermediate 1 using 1-chloro-2-iodobenzene in place of 1-iodo-2-methylbenzene.

### Intermediate 1/3: tert-Butyl 6-bromo-2-(2-(trifluoromethyl)phenyl)-1H-indole-1-carboxylate (Int 1/3)

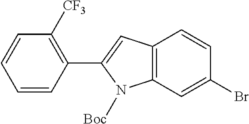 [SMILES: CC(C)(C)OC(=O)n1c(-c2ccccc2C(F)(F)F)cc2ccc(Br)cc21]

The title compound was prepared similar as described for Intermediate 1 using 1-iodo-2-(trifluoromethyl)benzene in place of 1-iodo-2-methylbenzene.

### Intermediate 1/4: tert-Butyl 6-bromo-2-(3-chlorophenyl)-1H-indole-1-carboxylate (Int 1/4)

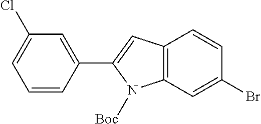 [SMILES: CC(C)(C)OC(=O)n1c(-c2cccc(Cl)c2)cc2ccc(Br)cc21]

The title compound was prepared similar as described for Intermediate 1 using 1-chloro-3-iodobenzene in place of 1-iodo-2-methylbenzene.

### Intermediate 1/5: tert-Butyl 6-bromo-2-(2,4-dichlorophenyl)-1H-indole-1-carboxylate (Int 1/5)

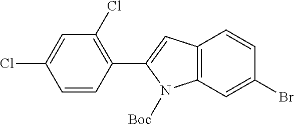 [SMILES: CC(C)(C)OC(=O)n1c(-c2ccc(Cl)cc2Cl)cc2ccc(Br)cc21]

The title compound was prepared similar as described for Intermediate 1 using 2,4-dichloro-1-iodobenzene in place of 1-iodo-2-methylbenzene.

### Intermediate 3: 2-(o-Tolyl)-1H-indol-6-amine (Int 3)

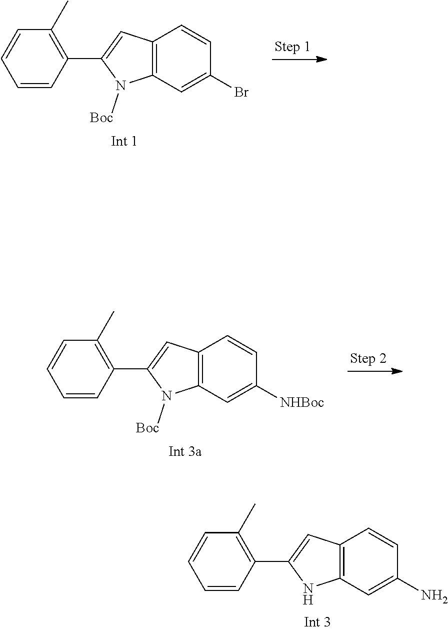 [SMILES: Cc1ccccc1-c1cc2ccc(Br)cc2n1C(=O)OC(C)(C)C ; Cc1ccccc1-c1cc2ccc(NC(=O)OC(C)(C)C)cc2n1C(=O)OC(C)(C)C ; Cc1ccccc1-c1cc2ccc(N)cc2[nH]1]

### Step 1: tert-Butyl 6-((tert-butoxycarbonyl)amino)-2-(o-tolyl)-1H-indole-1-carboxylate (Int 3a)

To a mixture of tert-butyl 6-bromo-2-(o-tolyl)-1H-indole-1-carboxylate (Int 1) (3.85 g, 10.0 mmol) in dioxane (40 mL) and water (4 mL) was added tert-butyl carbamate (1.41 g, 12.00 mmol), Cs₂CO₃ (4.88 g, 15.00 mmol), Xantphos (385 mg, 0.66 mmol) and Pd₂(dba)₃ (385 mg, 0.42 mmol). The mixture was stirred at 110° C. for 2 h under N₂. Water (80 mL) was added and the mixture was extracted with EtOAc (3×50 mL). The combined organic layers were washed with brine, dried over anhydrous Na₂SO₄ and filtered. The filtrate was concentrated to dryness and the residue was purified by column chromatography (PE/EtOAc=5:1) to give the title compound as a yellow solid.

### Step 2: 2-(o-Tolyl)-1H-indol-6-amine (Int 3)

A mixture of tert-butyl 6-((tert-butoxycarbonyl)amino)-2-(o-tolyl)-1H-indole-1-carboxylate (Int 3a) (2.45 g, 5.81 mmol), TFA (8 mL) and DCM (25 mL) was stirred at rt for 2 h. Water (30 mL) was added and the pH was adjust to pH=7 by adding NaHCO₃. The mixture was extracted with DCM (3×30 mL). The combined organic layers were dried over anhydrous Na₂SO₄, filtered and concentrated to dryness to give the title compound as a solid.

### Intermediate 4: 1-Methyl-1H-1,2,4-triazole-5-carboxamide (Int 4)

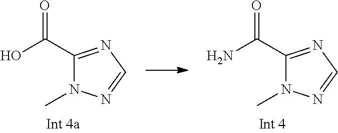

A mixture of 1-methyl-1H-1,2,4-triazole-5-carboxylic acid (7.0 g, 55.1 mmol) in SOCl₂ (20 mL) was heated to 70° C. for 2 h. The mixture was concentrated to dryness. The residue was dissolved in NH₃/MeOH (7M, 40 mL) and stirred at rt overnight. The precipitated solid was filtered off, extracted with Et₂O and dried under reduced pressure to give the title compound.

### Intermediate 4/1: 1-Isopropyl-1H-1,2,4-triazole-5-carboxamide (Int 4/1)

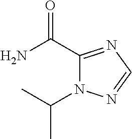

The title compound was prepared similar as described for Intermediate 4 using 1-isopropyl-1H-1,2,4-triazole-5-carboxylic acid in place of 1-methyl-1H-1,2,4-triazole-5-carboxylic acid.

### Intermediate 5: 4-Chloro-2-ethynyl-1-(trifluoromethyl)benzene (Int 5)

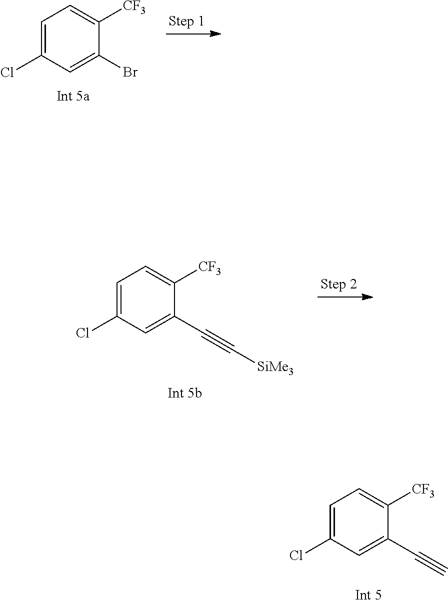 [SMILES: FC(F)(F)c1ccc(Cl)cc1Br ; C[Si](C)(C)C#Cc1cc(Cl)ccc1C(F)(F)F ; C#Cc1cc(Cl)ccc1C(F)(F)F]

### Step 1: ((5-Chloro-2-(trifluoromethyl)phenyl)ethynyl)trimethylsilane (Int 5b)

A mixture of Pd(PPh₃)₄ (2.20 g, 1.94 mmol), CuI (0.74 g, 3.88 mmol), 2-bromo-4-chloro-1-(trifluoromethyl)benzene (Int 5a) (10.00 g, 38.76 mmol) and ethynyltrimethylsilane (13.30 g, 135.66 mmol) in TEA was stirred at 70° C. overnight. The mixture was concentrated, EtOAc (200 mL) was added and the mixture was filtered through celite. The mixture was concentrated to dryness and the residue was purified by column chromatography (Hexane) to give the title compound as a yellow oil.

### Step 2: 4-Chloro-2-ethynyl-1-(trifluoromethyl)benzene (Int 5)

To a solution of ((5-chloro-2-(trifluoromethyl)phenyl)ethynyl)trimethylsilane (Int 5b) (10.00 g, 36.20 mmol) in MeOH (15 mL) was added K₂CO₃ (10.00 g, 72.40 mmol) and the mixture was stirred at rt for 0.5 h. The mixture was poured into ice-water and extracted with diethyl ether (2×100 mL). The combined organic layers were dried over MgSO₄, filtered and concentrated to dryness to give the title compound.

### Intermediate 6: 1-(Difluoromethyl)-2-ethynyl-4-fluorobenzene (Int 6)

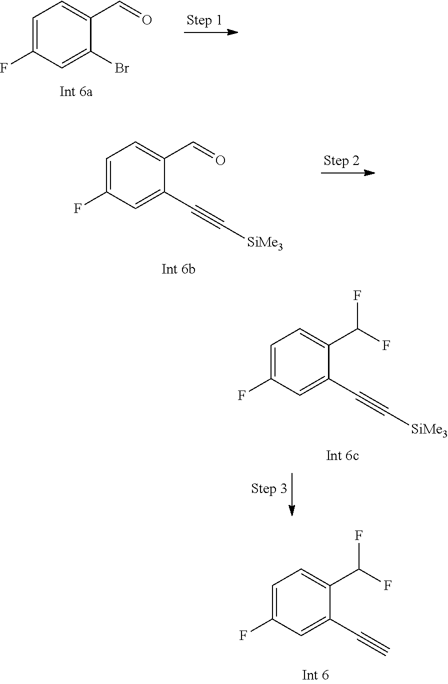 [SMILES: O=Cc1ccc(F)cc1Br ; C[Si](C)(C)C#Cc1cc(F)ccc1C=O ; C[Si](C)(C)C#Cc1cc(F)ccc1C(F)F ; C#Cc1cc(F)ccc1C(F)F]

### Step 1: 4-Fluoro-2-((trimethylsilyl)ethynyl)benzaldehyde (Int 6b)

The title compound was prepared similar as described for Intermediate 5b, step 1 using 2-bromo-4-fluorobenzaldehyde (Int 6a) in place of 2-bromo-4-chloro-1-(trifluoromethyl)benzene (Int 5a).

### Step 2: ((2-(Difluoromethyl)-5-fluorophenyl)ethynyl)trimethylsilane (Int 6c)

To a solution of 4-fluoro-2-((trimethylsilyl)ethynyl)benzaldehyde (Int 6b) (6.60 g, 30.00 mmol) in DCM (80 mL) was added DAST (9.66 g, 60.00 mmol) at 0° C. The mixture was stirred at rt for 4 h. The mixture was poured into ice-water and extracted with DCM (2×50 mL). The combined organic layers were dried over MgSO₄, filtered and concentrated to dryness. The residue was purified by column chromatography (gradient 5-30% EtOAc in PE) to give the title compound as yellow oil.

### Step 3: 1-(Difluoromethyl)-2-ethynyl-4-fluorobenzene (Int 6)

The title compound was prepared similar as described for Intermediate 5, step 3 using ((2-(difluoromethyl)-5-fluorophenyl)ethynyl)trimethylsilane (Int 6c) in place of ((5-chloro-2-(trifluoromethyl)phenyl)ethynyl)trimethylsilane (Int 5b).

### Intermediate 6/1: 1-(Difluoromethyl)-2-ethynylbenzene (Int 6/1)

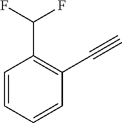

The title compound was prepared similar as described for Intermediate 6 using in step 1 2-bromobenzaldehyde in place of 2-bromo-4-fluorobenzaldehyde (Int 6a).

### Intermediate 7: tert-Butyl 6-amino-5-methyl-2-(2-(trifluoromethyl)phenyl)-1H-indole-1-carboxylate (Int 2)

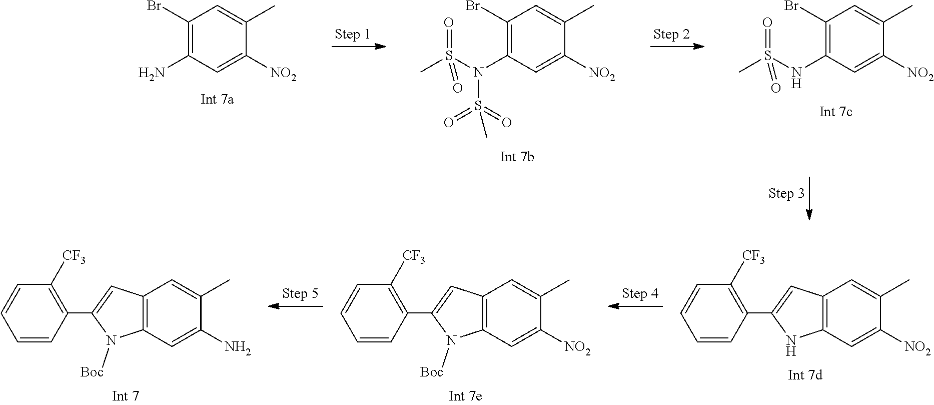 [SMILES: Cc1cc(Br)c(N)cc1[N+](=O)[O-] ; Cc1cc(Br)c(N(S(C)(=O)=O)S(C)(=O)=O)cc1[N+](=O)[O-] ; Cc1cc(Br)c(NS(C)(=O)=O)cc1[N+](=O)[O-] ; Cc1cc2cc(-c3ccccc3C(F)(F)F)n(C(=O)OC(C)(C)C)c2cc1N ; Cc1cc2cc(-c3ccccc3C(F)(F)F)n(C(=O)OC(C)(C)C)c2cc1[N+](=O)[O-] ; Cc1cc2cc(-c3ccccc3C(F)(F)F)[nH]c2cc1[N+](=O)[O-]]

### Step 1: N-(2-bromo-4-methyl-5-nitrophenyl)-N-(methylsulfonyl)methanesulfonamide (Int 7b)

Methanesulfonyl chloride (5.25 g, 45.7 mmol) was added dropwise to a solution of 2-bromo-4-methyl-5-nitroaniline (Int 7a) (3.00 g, 13.0 mmol) and TEA (4.61 g, 45.7 mmol) in DCM (50 mL) at 0° C. The mixture was allowed to warm to rt and stirred overnight.

The mixture was concentrated to dryness and the residue was purified by column chromatography (gradient 5-100% EtOAc in DCM) to give the title compound as a yellow solid.

### Step 2: N-(2-bromo-4-methyl-5-nitrophenyl)methanesulfonamide (Int 7c)

N-(2-Bromo-4-methyl-5-nitrophenyl)-N-(methylsulfonyl)methanesulfonamide (Int 7b) (4.07 g, 10.6 mmol) was dissolved in a mixture of aqueous NaOH solution (10 w/w %, 30 mL) and tetrahydrofuran (30 mL). The mixture was stirred at rt for 16 h. The mixture was concentrated, water was added and the mixture was acidified to pH=4 using an aqueous citric acid solution. The precipitated solid was filtered and dried to give the title compound as a yellow solid.

### Step 3: 5-Methyl-6-nitro-2-(2-(trifluoromethyl)phenyl)-1H-indole (Int 7d)

A mixture of N-(2-bromo-4-methyl-5-nitrophenyl)methanesulfonamide (Int 7c) (3.02 g, 9.82 mmol), 1-ethynyl-2-(trifluoromethyl)benzene (1.67 g, 9.82 mmol), bis(triphenylphosphine)palladium(II) dichloride (337 mg, 0.48 mmol), copper(I) iodide (92 mg, 0.48 mmol) and triethylamine (4.37 g, 43.25 mmol) in DMF (30 mL) was stirred at 100° C. for 3 h. DBU (3 mL) was added and the mixture was stirred at 100° C. overnight. The mixture was cooled to rt. Aqueous NH₄Cl was added and the mixture was extracted with EtOAc. The combined organic layers were dried over anhydrous MgSO₄, filtered and concentrated to dryness. The residue was purified by column chromatography (gradient 5-100% EtOAc in PE) to give the title compound as a yellow solid.

### Step 4: tert-Butyl 5-methyl-6-nitro-2-(2-(trifluoromethyl)phenyl)-1H-indole-1-carboxylate (Int 7e)

To a mixture of 5-methyl-6-nitro-2-(2-(trifluoromethyl)phenyl)-1H-indole (Int 7d) (2.54 g, 7.95 mmol) in DCM (40 mL) a solution of di-tert-butyl dicarbonate (2.10 g, 9.60 mmol) in DCM (15 mL) was added followed by DMAP (200 mg). The mixture was stirred at rt for 2 h. The mixture was absorbed onto silica and purified by column chromatography (DCM/EtOAc=9:1) to give the title compound as a white solid.

### Step 5: tert-Butyl 6-amino-5-methyl-2-(2-(trifluoromethyl)phenyl)-1H-indole-1-carboxylate (Int 7)

To a mixture of tert-butyl 5-methyl-6-nitro-2-(2-(trifluoromethyl)phenyl)-1H-indole-1-carboxylate (Int 7e) (2.94 g, 7.00 mmol) in EtOH (30 mL) and H₂O (15 mL), NH₄Cl (3.78 g, 70 mmol) and Fe powder (3.92 g, 70 mmol) were added. The mixture was stirred at rt overnight. The mixture was absorbed onto silica and purified by column chromatography (DCM/EtOAc=9:1) to give the title compound as a yellow solid.

### Intermediate 8: tert-Butyl 6-amino-2-(2-(difluoromethyl)phenyl)-1H-indole-1-carboxylate (Int 8)

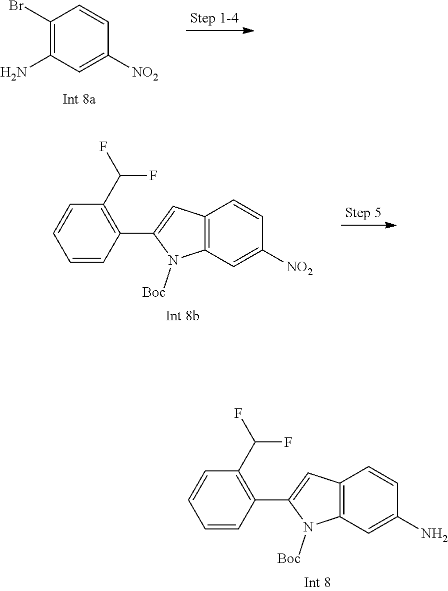 [SMILES: Nc1cc([N+](=O)[O-])ccc1Br ; CC(C)(C)OC(=O)n1c(-c2ccccc2C(F)F)cc2ccc([N+](=O)[O-])cc21 ; CC(C)(C)OC(=O)n1c(-c2ccccc2C(F)F)cc2ccc(N)cc21]

### Steps 1-4: tert-Butyl 2-(2-(difluoromethyl)phenyl)-6-nitro-1H-indole-1-carboxylate (Int 8b)

The title compound was prepared similar as described for Intermediate 7e, steps 1 to 4 using in step 1 2-bromo-5-nitroaniline (Int 8a) in place of bromo-4-methyl-5-nitroaniline (Int 7a) and in step 3 1-(difluoromethyl)-2-ethynylbenzene (Int 6/1) in place of 1-ethynyl-2-(trifluoromethyl)benzene.

### Step 5: tert-Butyl 6-amino-2-(2-(difluoromethyl)phenyl)-1H-indole-1-carboxylate (Int 8)

A mixture of tert-butyl 2-(2-(difluoromethyl)phenyl)-6-nitro-1H-indole-1-carboxylate (Int 8b) (776 mg, 2.00 mmol), Zn powder (1.30 g, 20.0 mmol) and NH₄Cl (1.06 g, 20.0 mmol) in THF/MeOH/H₂O (5/5/10 mL) was stirred at 50° C. for 2 h. The mixture was cooled to rt and filtered through celite. The mixture was concentrated to dryness and the residue was purified by column chromatography (gradient 0-50% EtOAc in PE) to give the title compound as a yellow oil.

### Intermediates 8/1 to 8/2

The following Intermediates were prepared similar as described for Intermediate 8 using the appropriate building blocks.

| | building block | |
|---|---|---|
| Int. # | Step 3 | Structure |
| Int 8/1 | 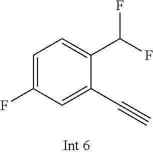 | 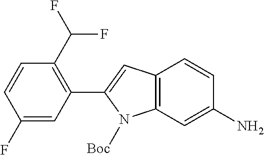 [SMILES: CC(C)(C)OC(=O)n1c(-c2cc(F)ccc2C(F)F)cc2ccc(N)cc21] |
| Int 8/2 | 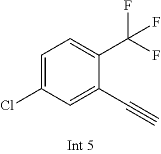 | 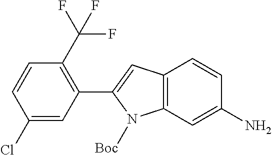 [SMILES: CC(C)(C)OC(=O)n1c(-c2cc(Cl)ccc2C(F)(F)F)cc2ccc(N)cc21] |

### Intermediate 9: tert-Butyl 6-amino-3-chloro-2-(2-(difluoromethyl)phenyl)-1H-indole-1-carboxylate (Int 9)

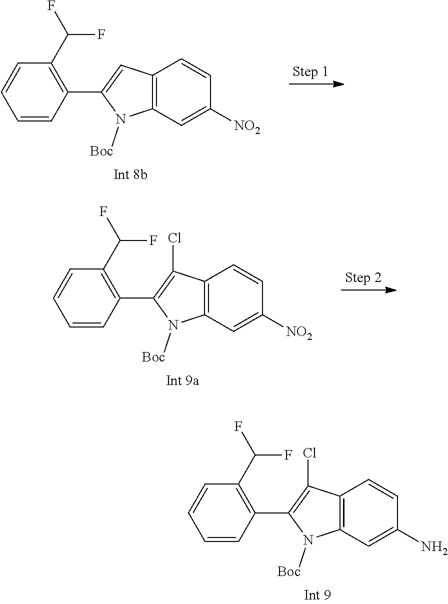 [SMILES: CC(C)(C)OC(=O)n1c(-c2ccccc2C(F)F)cc2ccc([N+](=O)[O-])cc21 ; CC(C)(C)OC(=O)n1c(-c2ccccc2C(F)F)c(Cl)c2ccc([N+](=O)[O-])cc21 ; CC(C)(C)OC(=O)n1c(-c2ccccc2C(F)F)c(Cl)c2ccc(N)cc21]

### Step 1: tert-Butyl 3-chloro-2-(2-(difluoromethyl)phenyl)-6-nitro-1H-indole-1-carboxylate (Int 9a)

A mixture of tert-butyl 2-(2-(difluoromethyl)phenyl)-6-nitro-1H-indole-1-carboxylate (Int 8b) (388 mg, 1.00 mmol) and NCS (160 mg, 1.20 mmol) in DMF (4 mL) was stirred at rt overnight. The mixture was poured into ice-water and extracted with EtOAc (2×20 mL). The combined organic layers were dried over MgSO₄, filtered and concentrated to dryness. The residue was purified by silica gel chromatography (gradient 0-50% EtOAc in PE) to give the title compound as yellow solid.

### Step 2: tert-Butyl 6-amino-3-chloro-2-(2-(difluoromethyl)phenyl)-1H-indole-1-carboxylate (Int 9)

A mixture of tert-butyl 3-chloro-2-(2-(difluoromethyl)phenyl)-6-nitro-1H-indole-1-carboxylate (Int 9a) (300 mg, 0.71 mmol), Zn powder (462 mg, 7.10 mmol) and NH₄Cl (376 mg, 7.10 mmol) in THF/MeOH/H₂O (5/5/10 mL) was stirred at 50° C. for 2 h. The reaction was cooled to rt. The mixture was filtered through celite. The mixture was concentrated to dryness and the residue was purified by column chromatography (gradient 0-50% EtOAc in PE) to give the title compound as a yellow oil.

### EXAMPLE 1: 1-METHYL-N-(2-(O-TOLYL)-1H-INDOL-6-YL)-1H-PYRAZOLE-5-CARBOXAMIDE (1)

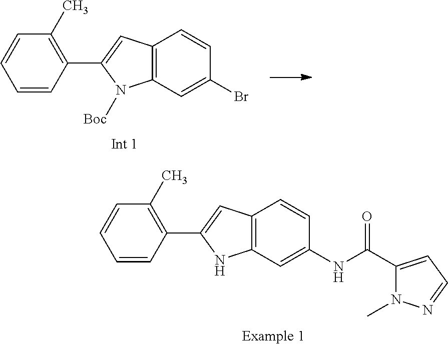 [SMILES: Cc1ccccc1-c1cc2ccc(Br)cc2n1C(=O)OC(C)(C)C ; Cc1ccccc1-c1cc2ccc(NC(=O)c3ccnn3C)cc2[nH]1]

A mixture of tert-butyl 6-bromo-2-(o-tolyl)-1H-indole-1-carboxylate (Int 1) (250 mg, 0.64 mmol), 1-methyl-1H-pyrazole-5-carboxamide (240 mg, 1.93 mmol), Pd₂(dba)₃ (117 mg, 0.13 mmol), Xantphos (115 mg, 0.26 mmol) and t-BuONa (241 mg, 1.93 mmol) in 1,4-dioxane (30 mL) was stirred at 110° C. under N₂ atmosphere for 5 h. The mixture was concentrated to dryness. The residue was purified by column chromatography (PE/EtOAc=7:3) to give a crude product which was purified by prep-HPLC to afford the title compound as a white solid. ¹H NMR (400 MHz, CD₃OD): δ ppm 7.96 (s, 1H), 7.56-7.52 (m, 3H), 7.32-7.26 (m, 3H), 7.19-7.16 (m, 1H), 6.99 (s, 1H), 6.54 (s, 1H), 4.19 (s, 3H), 2.52 (s, 3H). MS (ESI): 331.0 m/z [M+H]⁺.

### EXAMPLE 1/1: N-(2-(2-FLUOROPHENYL)-1H-INDOL-6-YL)-1-METHYL-1H-PYRAZOLE-5-CARBOXAMIDE (1/1)

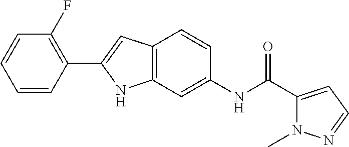 [SMILES: Cn1nccc1C(=O)Nc1ccc2cc(-c3ccccc3F)[nH]c2c1]

The title compound was prepared similar as described for Example 1 using tert-butyl 6-bromo-2-(2-fluorophenyl)-1H-indole-1-carboxylate (Int 1/1) in place of tert-butyl 6-bromo-2-(o-tolyl)-1H-indole-1-carboxylate (Int 1). ¹H NMR (400 MHz, DMSO-d₆): δ ppm 11.51 (s, 1H), 10.22 (s, 1H), 8.11 (s, 1H), 7.93-7.89 (m, 1H), 7.56-7.52 (m, 2H), 7.37-7.27 (m, 4H), 7.08 (s, 1H), 6.90 (s, 1H), 4.12 (s, 3H). MS (ESI): 335.1 m/z [M+H]⁺.

### EXAMPLE 1/2: N-(2-(3-CHLOROPHENYL)-1H-INDOL-6-YL)-1-METHYL-1H-PYRAZOLE-5-CARBOXAMIDE (1/2)

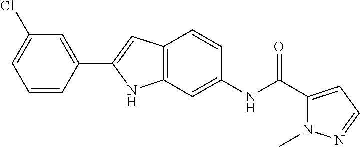 [SMILES: Cn1nccc1C(=O)Nc1ccc2cc(-c3cccc(Cl)c3)[nH]c2c1]

The title compound was prepared similar as described for Example 1 using tert-butyl 6-bromo-2-(3-chlorophenyl)-1H-indole-1-carboxylate (Int 1/4) in place of tert-butyl 6-bromo-2-(o-tolyl)-1H-indole-1-carboxylate (Int 1). ¹H NMR (400 MHz, DMSO-d₆): δ ppm 8.42 (br s, 1H), 8.12 (s, 1H), 7.76 (s, 1H), 7.64-6.32 (m, 1H), 7.63 (d, J=8.4 Hz, 1H), 7.53-7.51 (m, 2H), 7.38 (dd, J₁=J₂=8.0 Hz, 1H), 7.30-7.26 (m, 1H), 6.96 (dd, J₁=8.0, J₂=2.0 Hz, 1H), 6.82 (d, J=1.6 Hz, 1H), 6.68 (s, 1H), 4.25 (s, 3H). MS (ESI): m/z 351.1 [M+H]⁺.

### EXAMPLE 2: N-(2-(2-CHLOROPHENYL)-1H-INDOL-6-YL)-1-METHYL-1H-PYRAZOLE-5-CARBOXAMIDE (2)

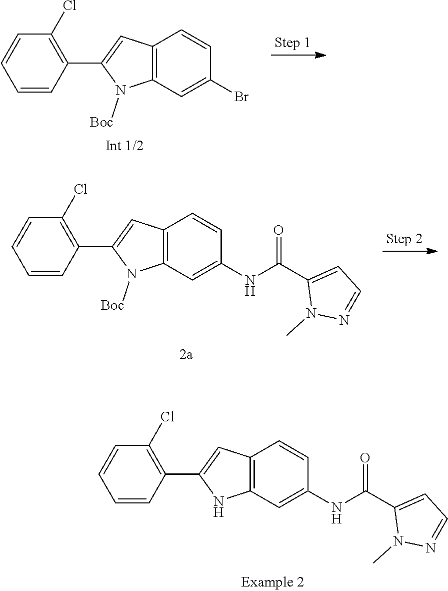 [SMILES: CC(C)(C)OC(=O)n1c(-c2ccccc2Cl)cc2ccc(Br)cc21 ; Cn1nccc1C(=O)Nc1ccc2cc(-c3ccccc3Cl)n(C(=O)OC(C)(C)C)c2c1 ; Cn1nccc1C(=O)Nc1ccc2cc(-c3ccccc3Cl)[nH]c2c1]

### Step 1: tert-Butyl 2-(2-chlorophenyl)-6-(1-methyl-1H-pyrazole-5-carboxamido)-1H-indole-1-carboxylate (2a)

A mixture of tert-butyl 6-bromo-2-(2-chlorophenyl)-1H-indole-1-carboxylate (Int 1/2) (400 mg, 0.98 mmol), 1-methyl-1H-pyrazole-5-carboxamide (184 mg, 1.47 mmol), Pd₂(dba)₃ (183 mg, 0.20 mmol), Xantphos (168 mg, 0.29 mmol) and Cs₂CO₃ (796 mg, 2.45 mmol) in 1,4-dioxane (30 mL) was stirred at 110° C. for 2 h. The mixture was concentrated to dryness and the residue was purified by column chromatography (PE/EtOAc=5:1) to give the title compound as a white solid.

### Step 2: N-(2-(2-Chlorophenyl)-1H-indol-6-yl)-1-methyl-1H-pyrazole-5-carboxamide (2)

To a mixture of tert-butyl 2-(2-chlorophenyl)-6-(1-methyl-1H-pyrazole-5-carboxamido)-1H-indole-1-carboxylate (2a) (300 mg, 0.67 mmol) in DCM (4 mL) was added dropwise TFA (2 mL). The mixture was stirred at rt for 2 h. Aqueous NaHCO₃ (10 mL) was added and the mixture was extracted with EtOAc. The organic layer was concentrated to dryness and the residue was purified by prep-HPLC to give the title compound as a white solid. ¹H NMR (400 MHz, DMSO-d₆): δ ppm 11.45 (s, 1H), 10.21 (s, 1H), 8.08 (s, 1H)), 7.74 (d, J=8.0 Hz, 1H), 7.60 (d, J=8.0 Hz, 1H), 7.57-7.52 (m, 2H), 7.48 (dd, J₁=J₂=7.2 Hz, 1H), 7.38 (dd, J₁=J₂=7.6 Hz, 1H), 7.29 (d, J=8.4 Hz, 1H), 7.08 (s, 1H), 6.88 (s, 1H), 4.12 (s, 3H). MS (ESI): 351.0 m/z [M+H]⁺.

### EXAMPLE 2/1: 1-METHYL-N-(2-(2-(TRIFLUOROMETHYL)PHENYL)-1H-INDOL-6-YL)-1H-PYRAZOLE-5-CARBOXAMIDE (2/1)

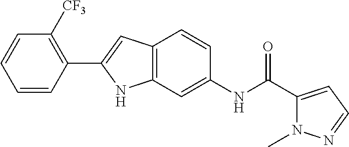 [SMILES: Cn1nccc1C(=O)Nc1ccc2cc(-c3ccccc3C(F)(F)F)[nH]c2c1]

The title compound was prepared similar as described for Example 2 using in step 1 tert-butyl 6-bromo-2-(2-(trifluoromethyl)phenyl)-1H-indole-1-carboxylate (Int 1/3) in place of tert-butyl 6-bromo-2-(2-chlorophenyl)-1H-indole-1-carboxylate (Int 1/2). ¹H NMR (400 MHz, DMSO-d₆): δ ppm 11.42 (s, 1H), 10.18 (s, 1H), 8.05 (s, 1H), 7.89 (d, J=7.6 Hz, 1H), 7.79 (dd, J₁=J₂=7.6 Hz, 1H), 7.71 (d, J=7.6 Hz, 1H), 7.64 (dd J₁=J₂=7.6 Hz, 1H), 7.56-7.51 (m, 2H), 7.29 (d, J=7.2 Hz, 1H), 7.08 (s, 1H), 6.54 (s, 1H), 4.11 (s, 3H). MS (ESI): 385.1 m/z [M+H]⁺.

### EXAMPLE 2/2: N-(2-(2,4-DICHLOROPHENYL)-1H-INDOL-6-YL)-1-METHYL-1H-PYRAZOLE-5-CARBOXAMIDE (2/2)

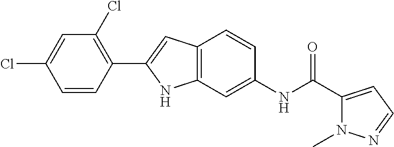 [SMILES: Cn1nccc1C(=O)Nc1ccc2cc(-c3ccc(Cl)cc3Cl)[nH]c2c1]

The title compound was prepared similar as described for Example 2 using in step 1 tert-butyl 6-bromo-2-(2,4-dichlorophenyl)-1H-indole-1-carboxylate (Int 1/5) in place of tert-butyl 6-bromo-2-(2-chlorophenyl)-1H-indole-1-carboxylate (Int 1/2). ¹H NMR (400 MHz, DMSO-d₆): δ ppm 11.49 (s, 1H), 10.20 (s, 1H), 8.08 (s, 1H), 7.77-7.74 (m, 2H), 7.59-7.54 (m, 3H), 7.29 (d, J=8.8 Hz, 1H), 7.08 (s, 1H), 6.92 (s, 1H), 4.11 (s, 3H). MS (ESI): m/z 385.1 [M+H]⁺.

### EXAMPLE 2/3: 1-ISOPROPYL-N-(2-(O-TOLYL)-1H-INDOL-6-YL)-1H-PYRAZOLE-5-CARBOXAMIDE (2/3)

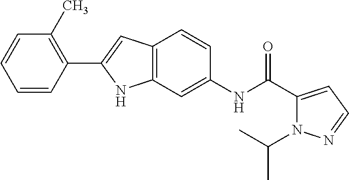 [SMILES: Cc1ccccc1-c1cc2ccc(NC(=O)c3ccnn3C(C)C)cc2[nH]1]

The title compound was prepared similar as described for Example 2 using in step 1 tert-butyl 6-bromo-2-(o-tolyl)-1H-indole-1-carboxylate (Int 1) in place of tert-butyl 6-bromo-2-(2-chlorophenyl)-1H-indole-1-carboxylate (Int 1/2) and 1-isopropyl-1H-pyrazole-5-carboxamide (Int 4/1) in place of 1-methyl-1H-pyrazole-5-carboxamide. ¹H NMR (400 MHz, DMSO-d₆): δ ppm 11.25 (s, 1H), 10.19 (s, 1H), 8.04 (s, 1H), 7.57-7.49 (m, 3H), 7.35-7.25 (m, 4H), 6.98 (s, 1H), 6.55 (s, 1H), 5.46-5.41 (m, 1H), 2.48 (s, 3H), 1.43 (d, J=5.2 Hz, 6H). (ESI): m/z 359.0 [M+H]⁺.

### EXAMPLE 2/4: 1-METHYL-N-(2-(2-(TRIFLUOROMETHYL)PHENYL)-1H-INDOL-6-YL)-1H-1,2,4-TRIAZOLE-5-CARBOXAMIDE (2/4)

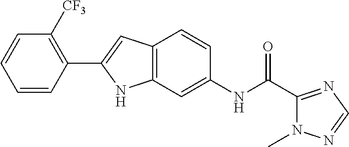 [SMILES: Cn1ncnc1C(=O)Nc1ccc2cc(-c3ccccc3C(F)(F)F)[nH]c2c1]

The title compound was prepared similar as described for Example 2 using in step 1 tert-butyl 6-bromo-2-(2-(trifluoromethyl)phenyl)-1H-indole-1-carboxylate (Int 1/3) in place of tert-butyl 6-bromo-2-(2-chlorophenyl)-1H-indole-1-carboxylate (Int 1/2) and 1-methyl-1H-1,2,4-triazole-5-carboxamide (Int 4) in place of 1-methyl-1H-pyrazole-5-carboxamide. ¹H NMR (500 MHz, DMSO-d₆): δ ppm 11.47 (s, 1H), 10.69 (s, 1H), 8.17 (s, 1H), 8.13 (s, 1H), 7.90 (d, J=7.5 Hz, 1H), 7.79-7.77 (m, 1H), 7.71-7.70 (m, 1H), 7.66-7.63 (m, 1H), 7.77 (d, J=7.5 Hz), 7.42-7.40 (m, 1H), 6.55 (s, 1H), 4.21 (m, 3H). (ESI): m/z 385.9 [M+H]⁺.

### EXAMPLE 2/5: N-(2-(2-(TRIFLUOROMETHYL)PHENYL)-1H-INDOL-6-YL)PICOLINAMIDE (2/5)

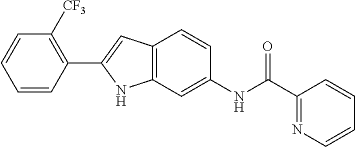 [SMILES: O=C(Nc1ccc2cc(-c3ccccc3C(F)(F)F)[nH]c2c1)c1ccccn1]

The title compound was prepared similar as described for Example 2 using in step 1 tert-butyl 6-bromo-2-(2-(trifluoromethyl)phenyl)-1H-indole-1-carboxylate (Int 1/3) in place of tert-butyl 6-bromo-2-(2-chlorophenyl)-1H-indole-1-carboxylate (Int 1/2) and picolinamide in place of 1-methyl-1H-pyrazole-5-carboxamide. ¹H NMR (500 MHz, DMSO-d₆): δ ppm 11.46 (s, 1H), 10.59 (s, 1H), 8.77-8.75 (m, 1H), 8.28 (s, 1H), 8.18 (d, J=8.0 Hz, 1H), 8.10-8.07 (m, 1H), 7.80-7.64 (m, 4H), 7.54 (d, J=8.5 Hz, 1H), 7.43-7.41 (m, 1H), 6.55 (s, 1H). (ESI): m/z 381.9 [M+H]⁺.

### EXAMPLE 3: N-(2-(O-TOLYL)-1H-INDOL-6-YL)-1H-PYRAZOLE-5-CARBOXAMIDE (3)

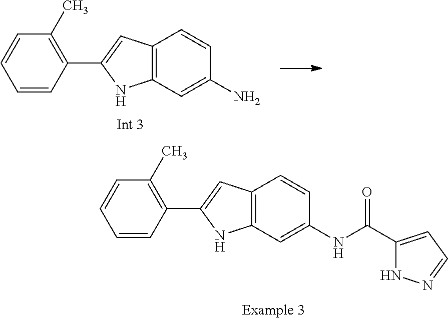 [SMILES: Cc1ccccc1-c1cc2ccc(N)cc2[nH]1 ; Cc1ccccc1-c1cc2ccc(NC(=O)c3ccn[nH]3)cc2[nH]1]

A mixture of 1H-pyrazole-5-carboxylic acid (227 mg, 2.00 mmol), 2-(o-tolyl)-1H-indol-6-amine (Int 3) (300 mg, 1.35 mmol), HATU (770 mg, 2.00 mmol), DIEA (0.7 mL, 4.05 mmol) and DMF (20 mL) was stirred at rt overnight. The mixture was concentrated to dryness and the residue was purified by prep-HPLC to give the title compound as a white solid. ¹H NMR (400 MHz, DMSO-d₆): δ ppm 13.38 (s, 1H), 11.24 (s, 1H), 9.90 (s, 1H), 8.15 (s, 1H), 7.90 (s, 1H), 7.56-7.54 (m, 1H), 7.47 (d, J=4.4 Hz, 1H), 7.34-7.25 (m, 4H), 6.79 (s, 1H), 6.54 (s, 1H), 2.48 (s, 3H). MS (ESI): m/z 317.2 [M+H]⁺.

### EXAMPLE 3/1: 1-METHYL-N-(2-(O-TOLYL)-1H-INDOL-6-YL)-1H-1,2,4-TRIAZOLE-5-CARBOXAMIDE (3/1)

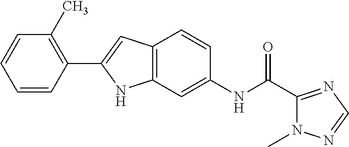 [SMILES: Cc1ccccc1-c1cc2ccc(NC(=O)c3ncnn3C)cc2[nH]1]

The title compound was prepared similar as described for Example 3 using 1-methyl-1H-1,2,4-triazole-5-carboxylic acid in place of 1H-pyrazole-5-carboxylic acid. ¹H NMR (400 MHz, DMSO-d₆): δ ppm 11.31 (s, 1H), 10.67 (s, 1H), 8.17-8.12 (m, 2H), 7.56-7.50 (m, 2H), 7.39-7.29 (m, 4H), 6.56 (s, 1H), 4.21 (s, 3H), 2.48 (s, 3H). MS (ESI): m/z 332.1 [M+H]⁺.

### EXAMPLE 3/2: 1-METHYL-N-(2-(O-TOLYL)-1H-INDOL-6-YL)-1H-PYRROLE-2-CARBOXAMIDE (3/2)

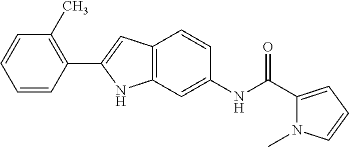 [SMILES: Cc1ccccc1-c1cc2ccc(NC(=O)c3cccn3C)cc2[nH]1]

The title compound was prepared similar as described for Example 3 using 1-methyl-1H-pyrrole-2-carboxylic acid in place of 1H-pyrazole-5-carboxylic acid. ¹H NMR (500 MHz, DMSO-d₆): δ ppm 11.17 (s, 1H), 9.70 (s, 1H), 8.03 (s, 1H), 7.55-7.53 (m, 1H), 7.47-7.45 (m, 1H), 7.34-7.23 (m, 4H), 7.04-6.98 (m, 2H), 6.52 (s, 1H), 6.09 (s, 1H), 3.90 (s, 3H), 2.48 (s, 3H). MS (ESI): m/z 330.0 [M+H]⁺.

### EXAMPLE 3/3: N-(2-(O-TOLYL)-1H-INDOL-6-YL)FURAN-2-CARBOXAMIDE (3/3)

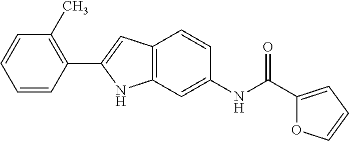 [SMILES: Cc1ccccc1-c1cc2ccc(NC(=O)c3ccco3)cc2[nH]1]

The title compound was prepared similar as described for Example 3 using furan-2-carboxylic acid in place of 1H-pyrazole-5-carboxylic acid. ¹H NMR (400 MHz, DMSO-d₆): δ ppm 11.27 (s, 1H), 10.12 (s, 1H), 8.08 (s, 1H), 7.93 (d, J=1.2 Hz, 1H), 7.56-7.54 (m, 1H), 7.49 (d, J=8.4 Hz, 1H), 7.34-7.27 (m, 5H), 6.71 (dd, J₁=2.0 Hz, J₂=3.6 Hz, 1H), 6.54 (d, J=1.6 Hz, 1H), 2.48 (s, 3H). MS (ESI): m/z 317.1 [M+H]⁺.

### EXAMPLE 3/4: 4-HYDROXY-N¹-(2-(O-TOLYL)-1H-INDOL-6-YL)ISOPHTHALAMIDE (3/4)

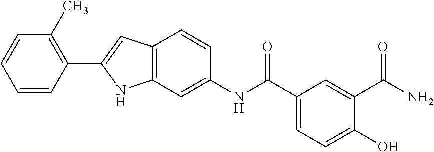 [SMILES: Cc1ccccc1-c1cc2ccc(NC(=O)c3ccc(O)c(C(N)=O)c3)cc2[nH]1]

The title compound was prepared similar as described for Example 3 using 3-carbamoyl-4-hydroxybenzoic acid in place of 1H-pyrazole-5-carboxylic acid. ¹H NMR (400 MHz, DMSO-d₆): δ ppm 13.40 (s, 1H), 11.25 (s, 1H), 10.02 (s, 1H), 8.55-8.51 (m, 2H), 8.10-8.02 (m, 3H), 7.57-7.49 (m, 2H), 7.35-7.26 (m, 4H), 7.01 (d, J=8.6 Hz, 1H), 6.55 (d, J=1.6 Hz, 1H), 2.49 (s, 3H). MS (ESI): m/z 386.1 [M+H]⁺.

### EXAMPLE 3/5: N-(2-(O-TOLYL)-1H-INDOL-6-YL)PICOLINAMIDE (3/5)

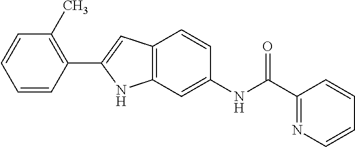 [SMILES: Cc1ccccc1-c1cc2ccc(NC(=O)c3ccccn3)cc2[nH]1]

The title compound was prepared similar as described for Example 3 using picolinic acid in place of 1H-pyrazole-5-carboxylic acid. ¹H NMR (400 MHz, DMSO-d₆): δ ppm 11.31 (s, 1H), 10.55 (s, 1H), 8.76 (d, J=4.0 Hz, 1H), 8.28 (s, 1H), 8.20-8.08 (m, 1H), 8.08-8.06 (m, 1H), 7.70-7.67 (m, 1H), 7.57-7.51 (m, 2H), 7.40-7.26 (m, 4H), 6.56 (d, J=0.8 Hz, 1H), 2.49 (s, 3H). MS (ESI): m/z 328.2 [M+H]⁺.

### EXAMPLE 4: 1-METHYL-N-(5-METHYL-2-(2-(TRIFLUOROMETHYL)PHENYL)-1H-INDOL-6-YL)-1H-1,2,4-TRIAZOLE-5-CARBOXAMIDE (4)

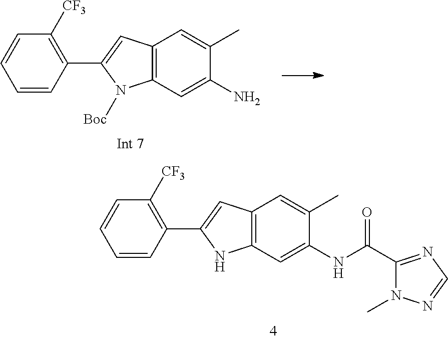 [SMILES: Cc1cc2cc(-c3ccccc3C(F)(F)F)n(C(=O)OC(C)(C)C)c2cc1N ; Cc1cc2cc(-c3ccccc3C(F)(F)F)[nH]c2cc1NC(=O)c1ncnn1C]

To a mixture of tert-butyl 6-amino-5-methyl-2-(2-(trifluoromethyl)phenyl)-1H-indole-1-carboxylate (Int 7) (500 mg, 1.28 mmol) and Et₃N (194 mg, 1.92 mmol) in THF (10 mL) 1-methyl-1H-1,2,4-triazole-5-carbonyl chloride (186 mg, 1.28 mmol) was added. The mixture was stirred at rt for 5 h. The mixture was diluted with DCM, filtered and concentrated to dryness. The residue was purified by column chromatography (gradient 5-25% EtOAc in PE) to afford a yellow solid. The solid was dissolved in DCM (5 mL), TFA (5 mL) was added and the mixture was stirred at rt overnight. The mixture was concentrated to dryness and the residue was purified by reverse phase chromatography (c18, gradient 25-55% acetonitrile/10 mM aqueous NH₄HCO₃) to give the title compound as a white solid. ¹H NMR (500 MHz, DMSO-d₆): δ ppm 11.37 (s, 1H), 10.08 (s, 1H), 8.16 (s, 1H), 7.89 (d, J=8.5 Hz, 1H), 7.80-7.77 (m, 1H), 7.77-7.63 (m, 3H), 7.45 (s, 1H), 6.50 (s, 1H), 4.21 (m, 3H), 2.34 (m, 3H). MS (ESI): m/z 400.1 [M+H]⁺.

### EXAMPLE 4/1: N-(5-METHYL-2-(2-(TRIFLUOROMETHYL)PHENYL)-1H-INDOL-6-YL)PICOLINAMIDE (4/1)

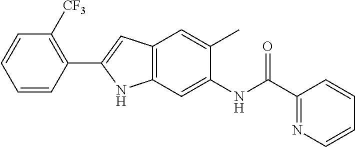 [SMILES: Cc1cc2cc(-c3ccccc3C(F)(F)F)[nH]c2cc1NC(=O)c1ccccn1]

The title compound was prepared similar as described for Example 5 using picolinoyl chloride in place of 1-methyl-1H-1,2,4-triazole-5-carbonyl chloride. ¹H NMR (500 MHz, DMSO-d₆): δ ppm 11.36 (s, 1H), 10.29 (s, 1H), 8.77-8.75 (m, 1H), 8.21 (d, J=8.0 Hz, 1H), 8.16 (s, 1H), 8.12-8.08 (m, 1H), 7.87 (d, J=8.0 Hz, 1H), 7.79-7.77 (m, 1H), 7.71-7.62 (m, 3H), 7.46 (s, 1H), 6.50 (s, 1H), 2.42 (s, 3H). MS (ESI): m/z 396.1 [M+H]⁺.

### EXAMPLE 5: N-(3-(TERT-BUTYL)-2-(O-TOLYL)-1H-INDOL-6-YL)-1-METHYL-1H-PYRAZOLE-5-CARBOXAMIDE (5)

Byproduct from an alternative synthesis of 1-methyl-N-(2-(o-tolyl)-1H-indol-6-yl)-1H-pyrazole-5-carboxamide (1)

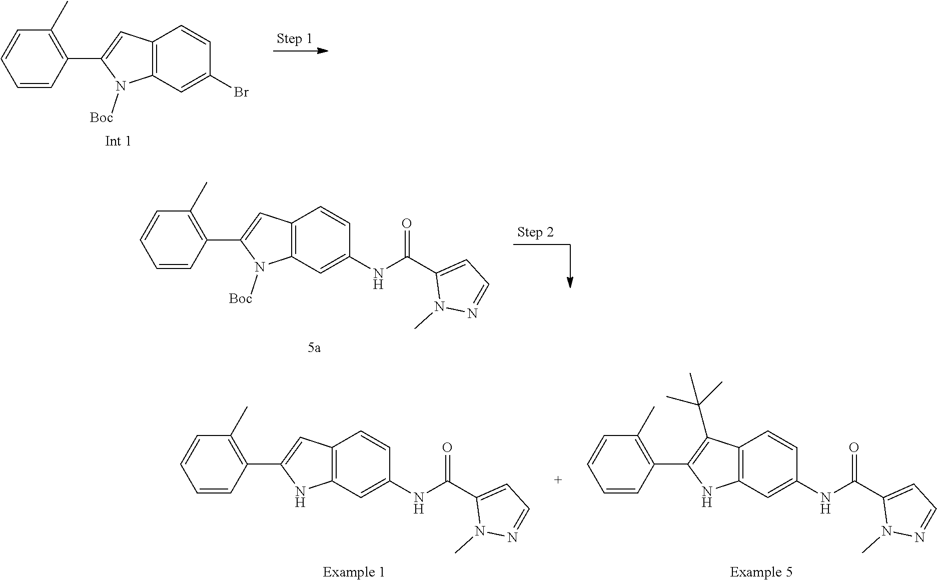 [SMILES: Cc1ccccc1-c1cc2ccc(Br)cc2n1C(=O)OC(C)(C)C ; Cc1ccccc1-c1cc2ccc(NC(=O)c3ccnn3C)cc2n1C(=O)OC(C)(C)C ; Cc1ccccc1-c1cc2ccc(NC(=O)c3ccnn3C)cc2[nH]1 ; Cc1ccccc1-c1[nH]c2cc(NC(=O)c3ccnn3C)ccc2c1C(C)(C)C]

### Step 1: tert-Butyl 6-(1-methyl-1H-pyrazole-5-carboxamido)-2-(o-tolyl)-1H-indole-1-carboxylate (5a)

To a solution of tert-butyl 6-bromo-2-(o-tolyl)-1H-indole-1-carboxylate (Int 1) (60.0 g, 156.0 mmol) in dioxane (800 mL) and water (80 mL) was added 1-methyl-1H-pyrazole-5-carboxamide (27.2 g, 216.0 mmol), Cs₂CO₃ (152.0 g, 46.8 mmol), Xantphos (18.0 g, 32.0 mmol) and Pd₂(dba)₃ (14.4 g, 16.00 mmol). The mixture was stirred at 110° C. for 2 h under N₂. Water (800 mL) was added and the mixture was extracted with EtOAc (3×400 mL). The combined organic layers were washed with brine, dried over anhydrous Na₂SO₄, filtered and concentrated to dryness. The residue was purified by silica gel column (PE/EtOAc=6:1) to give the title compound as a yellow solid.

### Step 2: 1-Methyl-N-(2-(o-tolyl)-1H-indol-6-yl)-1H-pyrazole-5-carboxamide (1) and N-(3-(tert-butyl)-2-(o-tolyl)-1H-indol-6-yl)-1-methyl-1H-pyrazole-5-carboxamide (5)

To a mixture of tert-butyl 6-(1-methyl-1H-pyrazole-5-carboxamido)-2-(o-tolyl)-1H-indole-1-carboxylate (5a) (34.0 g, 76.0 mmol) in DCM (300 mL) was added TFA (86.4 g, 760.0 mmol) and the mixture was stirred at 40° C. overnight. Saturated aqueous NaHCO₃ was added until pH=8 and the mixture was extracted with EtOAc (3×400 mL). The combined organic layers were washed with brine, dried over anhydrous Na₂SO₄, filtered and concentrated to dryness. The residue was purified by silica gel column (PE/EtOAc=3:1) to give the title compounds as solids. ¹H NMR of N-(3-(tert-butyl)-2-(o-tolyl)-1H-indol-6-yl)-1-methyl-1H-pyrazole-5-carboxamide (5) (500 MHz, DMSO-d₆): δ ppm 10.73 (s, 1H), 10.10 (s, 1H), 7.85 (s, 1H), 7.71 (J=8.5 Hz, 1H), 7.52 (s, 1H), 7.34-7.23 (m, 5H), 7.08 (s, 1H), 4.10 (s, 3H), 2.21 (s, 3H), 1.25 (s, 9H). MS (ESI): m/z 387.1 [M+H]⁺.

### EXAMPLE 6: N-(2-(2-(DIFLUOROMETHYL)PHENYL)-1H-INDOL-6-YL)-1-METHYL-1H-1,2,4-TRIAZOLE-5-CARBOXAMIDE (6)

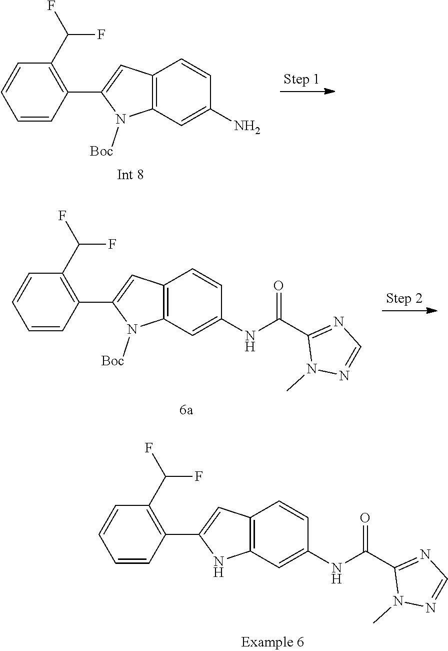 [SMILES: CC(C)(C)OC(=O)n1c(-c2ccccc2C(F)F)cc2ccc(N)cc21 ; Cn1ncnc1C(=O)Nc1ccc2cc(-c3ccccc3C(F)F)n(C(=O)OC(C)(C)C)c2c1 ; Cn1ncnc1C(=O)Nc1ccc2cc(-c3ccccc3C(F)F)[nH]c2c1]

### Step 1: tert-Butyl 2-(2-(Difluoromethyl)phenyl)-6-(1-methyl-1H-1,2,4-triazole-5-carboxamido)-1H-indole-1-carboxylate (6a)

A mixture of tert-butyl 6-amino-2-(2-(difluoromethyl)phenyl)-1H-indole-1-carboxylate (Int 8) (358 mg, 1.00 mmol), 1-methyl-1H-1,2,4-triazole-5-carboxylic acid (454 mg, 2.00 mmol), HATU (570 mg, 1.50 mmol) and TEA (202 mg, 2.00 mmol) in DMF (4 mL) was stirred at rt overnight. Water (10 mL) was added and the mixture was extracted with EtOAc (2×15 mL). The combined organic layers were dried over MgSO₄, filtered and concentrated to dryness. The residue was purified by silica gel chromatography (gradient 30-100% EtOAc in PE) to give the title compound as a yellow oil.

### Step 2: N-(2-(2-(Difluoromethyl)phenyl)-1H-indol-6-yl)-1-methyl-1H-1,2,4-triazole-5-carboxamide (6)

To a mixture of tert-butyl 2-(2-(difluoromethyl)phenyl)-6-(1-methyl-1H-1,2,4-triazole-5-carboxamido)-1H-indole-1-carboxylate (6a) (330 mg, 0.71 mmol) in DCM (1.5 mL) was added TFA (0.5 mL) and the mixture was stirred at rt overnight. The mixture was concentrated to dryness and the residue was purified by preparative HPLC to give the title compound as a yellow solid. 1H NMR (400 MHz, DMSO-d₆): δ ppm 11.58 (s, 1H), 10.73 (s, 1H), 8.17 (s, 2H), 7.82-7.67 (m, 3H), 7.66-7.53 (m, 2H), 7.44 (d, J=8.5 Hz, 1H), 7.14 (t, J=54.6 Hz, 1H), 6.57 (s, 1H), 4.21 (s, 3H). MS (ESI): m/z 368.1 [M+H]⁺.

### EXAMPLES 6/1 TO 6/6

The following Examples were prepared similar as described for Example 6 using the appropriate carboxamide building blocks and intermediates.

| | Building | | |
|---|---|---|---|
| # | blocks | Structure | Analytical data |
| 6/1 | 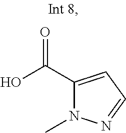 | 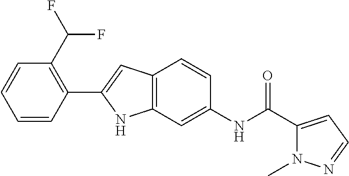 [SMILES: Cn1nccc1C(=O)Nc1ccc2cc(-c3ccccc3C(F)F)[nH]c2c1] | ¹H NMR (400 MHz, DMSO-d₆) δ: 11.54 (s, 1H), 10.21 (s, 1H), 8.09 (s, 1H), 7.81 (d, J = 7.8 Hz, 1H), 7.78-7.64 (m 2H), 7.60-7.53 (m, 3H), 7.34-7.04 (m, 3H), 6.56 (s, 1H), 4.12 (s, 3H). MS (ESI): m/z 367.1 [M + H]⁺. |
| 6/2 | 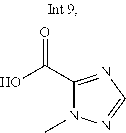 | 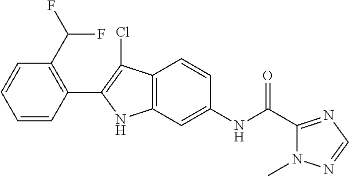 [SMILES: Cn1ncnc1C(=O)Nc1ccc2c(Cl)c(-c3ccccc3C(F)F)[nH]c2c1] | ¹H NMR (400 MHz, DMSO-d₆) δ: 11.79 (s, 1H), 10.82 (s, 1H), 8.18-8.16 (m, 2H), 7.87-7.79 (m, 1H), 7.79-7.65 (m, 2H), 7.64-7.54 (m, 2H), 7.50 (d, J = 8.6 Hz, 1H), 6.90 (t, J = 54.8 Hz, 1H), 4.21 (s, 3H). MS (ESI): m/z 402.0 [M + H]⁺. |
| 6/3 | 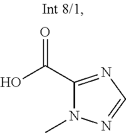 | 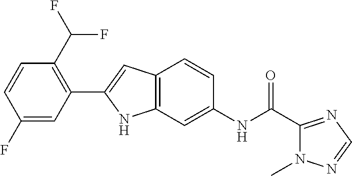 [SMILES: Cn1ncnc1C(=O)Nc1ccc2cc(-c3cc(F)ccc3C(F)F)[nH]c2c1] | ¹H NMR (400 MHz, DMSO- d₆): δ ppm 11.64 (s, 1H), 10.75 (s, 1H), 8.17 (s, 2H), 7.89-7.85 (m, 1H), 7.60-7.48 (m, 2H), 7.47-7.39 (m, 2H), 7.15 (t, J = 54.4 Hz, 1H), 6.64 (s, 1H), 4.21 (s, 3H). MS (ESI): m/z 386.1 [M + H]⁺. |
| 6/4 | 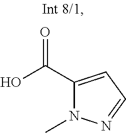 | 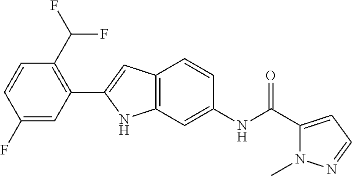 [SMILES: Cn1nccc1C(=O)Nc1ccc2cc(-c3cc(F)ccc3C(F)F)[nH]c2c1] | ¹H NMR (400 MHz, DMSO- d₆): δ ppm 11.60 (s, 1H), 10.23 (s, 1H), 8.09 (s, 1H), 7.89-7.85 (m, 1H), 7.60-7.54 (m, 3H), 7.44-7.02 (m, 4H), 6.63 (s, 1H), 4.12 (s, 3H). MS (ESI): m/z 385.0 [M + H]⁺. |
| 6/5 | 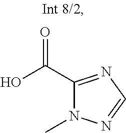 | 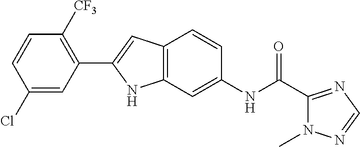 [SMILES: Cn1ncnc1C(=O)Nc1ccc2cc(-c3cc(Cl)ccc3C(F)(F)F)[nH]c2c1] | ¹H NMR (400 MHz, DMSO- d₆): δ ppm 11.57 (s, 1H), 10.72 (s, 1H), 8.17 (s, 1H), 8.13 (s, 1H), 7.92 (d, J = 8.6 Hz, 1H), 7.81 (s, 1H), 7.73- 7.70 (m, 1H), 7.56 (d, J = 8.6 Hz, 1H), 7.45-7.42 (m, 1H), 6.63 (s, 1H), 4.21 (s, 3H). MS (ESI): m/z 420.0 [M + H]⁺. |
| 6/6 | 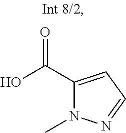 | 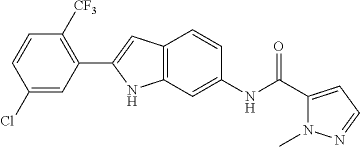 [SMILES: Cn1nccc1C(=O)Nc1ccc2cc(-c3cc(Cl)ccc3C(F)(F)F)[nH]c2c1] | ¹H NMR (400 MHz, DMSO-d₆) δ ppm 11.53 (s, 1H), 10.21 (s, 1H), 8.05 (s, 1H), 7.92 (d, J = 8.6 Hz, 1H), 7.80 (s, 1H), 7.71 (d, J = 9.4 Hz, 1H), 7.58-7.53 (m, 2H), 7.31 (d, J = 8.8 Hz, 1H), 7.09 (s, 1H), 6.63 (s, 1H), 4.11 (s, 3H). MS (ESI): m/z 419.0 [M + H]⁺. |

### Biological Assays

### AhR Direct Luciferase Reporter Assay in HepG2 Cells.

A stable cell line (HepG2 CYP1A1-LUC) was used in which part of the promoter region of the human CYP1A1 gene is stably integrated into the genome of human HepG2 hepatocytes (DSZM # ACC 180) in front of a Photinus pyralis Firefly Luciferase gene. A 1210 bp fragment comprising part of the human CYP1A1 promoter was isolated via SacI and BgIII restriction digestion from Lightswitch Clone S714555 (SwitchGearGenomics) and inserted between the SacI and BgIII sites in pGL4.30 (Promega # E8481) in front of the Firefly Luciferase gene. The resulting vector was linearized with NotI, transfected into HepG2 cells (DSMZ # ACC 180) and stably transfected clones selected with 250 μg/ml Hygromycin B. After repetitive rounds of subcloning and testing for robustly regulated luciferase activity after AhR agonist stimulation, a stable clonal HepG2 CYP1A1-Luc cell line was selected.

The HepG2 CYP1A1-Luc cells do express basal luciferase activity that can be increased via potent AhR agonists or decreased via potent AhR antagonists, added to the growth medium of the cells.

In typical reporter assays performed with this cell line, cells are grown in 96-well plates and AhR modulators are titrated into the growth medium in serial dilutions in RPMI-1640 Medium (Sigma # R7509) supplemented with 8.6% fetal calf serum (Sigma # F7524) and containing either no exogenous AhR agonist or 10 nM of the potent AhR agonist VAF347 (Calbiochem #182690). Cells are further cultivated for 18 hours and luciferase activities are determined from extracts of cells in buffers containing D-Luciferine and ATP using a LUMIstar Optima microplate Luminometer from BMG Labtech.

The AhR antagonistic potency of the example compounds is shown in Table 1 below (A=IC₅₀<100 nM, B=IC₅₀ 100 nM−1 μM, C=IC₅₀>1 μM).

| Example # | AhR potency |
|---|---|
| 1 | A |
| 1/1 | B |
| 1/2 | B |
| 2 | B |
| 2/1 | A |
| 2/2 | B |
| 2/3 | C |
| 2/4 | A |
| 2/5 | A |
| 3 | B |
| 3/1 | A |
| 3/2 | A |
| 3/3 | A |
| 3/4 | C |
| 3/5 | A |
| 4 | A |
| 4/1 | A |
| 5 | C |
| 6 | A |
| 6/1 | A |
| 6/3 | A |
| 6/4 | A |
| 6/5 | A |
| 6/6 | A |

1. A compound represented by Formula (I) 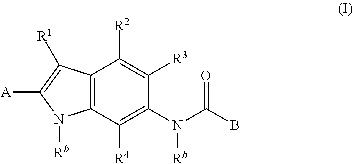
wherein
A is selected from 6- to 10-membered mono- or bicyclic aryl and 5- to 10-membered mono- or bicyclic heteroaryl containing 1 to 4 heteroatoms independently selected from N, O and S,
wherein aryl and heteroaryl are unsubstituted or substituted with 1 to 7 substituents independently selected from the group consisting of halogen, OH, CN, C₁₋₆-alkyl, O—C₁₋₆-alkyl, C(O)OR^{a}, OC(O)R^{a}, S(O)—C₁₋₆-alkyl, S(O)₂—C₁₋₆-alkyl, N(R^{a})₂, C(O)N(R^{a})₂, NR^{a}C(O)—C₁₋₆-alkyl, S(O)₂N(R^{a})₂, NR^{a}S(O)₂—C₁₋₆-alkyl and C₃₋₆-cycloalkyl,
wherein the alkyl and cycloalkyl are unsubstituted or substituted with 1 to 3 substituents independently selected from the group consisting of halogen, C₁₋₃-alkyl, halo-C₁₋₃-alkyl, OH, CN and oxo, or
wherein two substituents on the aryl or heteroaryl group together with the atoms they are attached to may form a 5- to 7-membered saturated or partially unsaturated carbocyclic ring or heterocyclic ring containing 1 to 3 heteroatoms independently selected from O, N and S,
wherein the carbocyclic or heterocyclic ring is unsubstituted or substituted with 1 to 5 substituents independently selected from the group consisting of halogen, C₁₋₆-alkyl and halo-C₁₋₆-alkyl;
B is selected from 6- to 10-membered mono- or bicyclic aryl and 5- to 10-membered mono- or bicyclic heteroaryl containing 1 to 4 heteroatoms independently selected from N, O and S,
wherein aryl and heteroaryl are unsubstituted or substituted with 1 to 7 substituents independently selected from the group consisting of halogen, OH, CN, C₁₋₆-alkyl, O—C₁₋₆-alkyl, C(O)OR^{a}, OC(O)R^{a}, S(O)—C₁₋₆-alkyl, S(O)₂—C₁₋₆-alkyl, N(R^{a})₂, C(O)N(R^{a})₂, NR^{a}C(O)—C₁₋₆-alkyl, S(O)₂N(R^{a})₂, NR^{a}S(O)₂—C₁₋₆-alkyl and C₃₋₆-cycloalkyl,
wherein the alkyl and cycloalkyl are unsubstituted or substituted with 1 to 3 substituents independently selected from the group consisting of halogen, C₁₋₃-alkyl, halo-C₁₋₃-alkyl, OH, CN and oxo, or
wherein two substituents on the aryl or heteroaryl group together with the atoms they are attached to may form a 5- to 7-membered saturated or partially unsaturated carbocyclic ring or heterocyclic ring containing 1 to 3 heteroatoms independently selected from O, N and S,
wherein the carbocyclic or heterocyclic ring is unsubstituted or substituted with 1 to 5 substituents independently selected from the group consisting of halogen, C₁₋₆-alkyl and halo-C₁₋₆-alkyl;
R¹, R², R³ and R⁴ are each independently selected from hydrogen, halogen, C₁₋₄-alkyl, halo-C₁₋₃-alkyl, OH, O—C₁₋₃-alkyl, and CN;
R^{a} is independently hydrogen or C₁₋₆-alkyl, and
R^{b} is independently hydrogen or C₁₋₆-alkyl.

2. A compound represented by Formula (I) 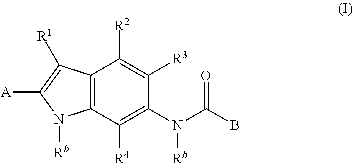
wherein
A is selected from 6- to 10-membered mono- or bicyclic aryl and 5- to 10-membered mono- or bicyclic heteroaryl containing 1 to 4 heteroatoms independently selected from N, O and S,
wherein aryl and heteroaryl are unsubstituted or substituted with 1 to 7 substituents independently selected from the group consisting of halogen, OH, CN, C₁₋₆-alkyl, O—C₁₋₆-alkyl, C(O)OR^{a}, OC(O)R^{a}, S(O)—C₁₋₆-alkyl, S(O)₂—C₁₋₆-alkyl, N(R^{a})₂, C(O)N(R^{a})₂, NR^{a}C(O)—C₁₋₆-alkyl, S(O)₂N(R^{a})₂, NR^{a}S(O)₂—C₁₋₆-alkyl and C₃₋₆-cycloalkyl,
wherein the alkyl and cycloalkyl are unsubstituted or substituted with 1 to 3 substituents independently selected from the group consisting of halogen, C₁₋₃-alkyl, halo-C₁₋₃-alkyl, OH, CN and oxo, or
wherein two substituents on the aryl or heteroaryl group together with the atoms they are attached to may form a 5- to 7-membered saturated or partially unsaturated carbocyclic ring or heterocyclic ring containing 1 to 3 heteroatoms independently selected from O, N and S,
wherein the carbocyclic or heterocyclic ring is unsubstituted or substituted with 1 to 5 substituents independently selected from the group consisting of is halogen, C₁₋₆-alkyl and halo-C₁₋₆-alkyl;
B is selected from 6- to 10-membered mono- or bicyclic aryl and 5- to 10-membered mono- or bicyclic heteroaryl containing 1 to 4 heteroatoms independently selected from N, O and S,
wherein aryl and heteroaryl are unsubstituted or substituted with 1 to 7 substituents independently selected from the group consisting of halogen, OH, CN, C₁₋₆-alkyl, O—C₁₋₆-alkyl, C(O)OR^{a}, OC(O)R^{a}, S(O)—C₁₋₆-alkyl, S(O)₂—C₁₋₆-alkyl, N(R^{a})₂, C(O)N(R^{a})₂, NR^{a}C(O)—C₁₋₆-alkyl, S(O)₂N(R^{a})₂, NR^{a}S(O)₂—C₁₋₆-alkyl and C₃₋₆-cycloalkyl,
wherein the alkyl and cycloalkyl are unsubstituted or substituted with 1 to 3 substituents independently selected from the group consisting of halogen, C₁₋₃-alkyl, halo-C₁₋₃-alkyl, OH, CN and oxo, or
wherein two substituents on the aryl or heteroaryl group together with the atoms they are attached to may form a 5- to 7-membered saturated or partially unsaturated carbocyclic ring or heterocyclic ring containing 1 to 3 heteroatoms independently selected from O, N and S,
wherein the carbocyclic or heterocyclic ring is unsubstituted or substituted with 1 to 5 substituents independently selected from the group consisting of halogen, C₁₋₆-alkyl and halo-C₁₋₆-alkyl;
R¹, R², R³ and R⁴ are each independently selected from hydrogen, halogen, C₁₋₄-alkyl, halo-C₁₋₃-alkyl, OH, O—C₁₋₃-alkyl, and CN;
R^{a} is independently hydrogen or C₁₋₆-alkyl, and
R^{b} is independently hydrogen or C₁₋₆-alkyl;
with the proviso that the compound is not methyl 4-(6-(4-(dimethylamino)benzamido)-1H-indol-2-yl)benzoate.

3. The compound of claim 1 or 2, wherein R^{b} is hydrogen.

4. The compound of at least one of claims 1 to 3, wherein A is selected from 6- to 10-membered mono- or bicyclic aryl and 5- to 10-membered mono- or bicyclic heteroaryl containing 1 to 4 heteroatoms independently selected from N, O and S,
wherein aryl and heteroaryl are unsubstituted or substituted with 1 to 7 substituents independently selected from the group consisting of halogen, OH, CN, C₁₋₆-alkyl, O—C₁₋₆-alkyl, C(O)OR^{a}, OC(O)R^{a}, S(O)—C₁₋₆-alkyl, S(O)₂—C₁₋₆-alkyl, N(R^{a})₂, C(O)N(R^{a})₂, NR^{a}C(O)—C₁₋₆-alkyl, S(O)₂N(R^{a})₂, NR^{a}S(O)₂—C₁₋₆-alkyl and C₃₋₆-cycloalkyl,
wherein the alkyl and cycloalkyl are unsubstituted or substituted with 1 to 3 substituents independently selected from the group consisting of halogen, C₁₋₃-alkyl, halo-C₁₋₃-alkyl, OH, CN and oxo, or
wherein two substituents on the aryl or heteroaryl group together with the atoms they are attached to may form a 5- to 7-membered saturated or partially unsaturated carbocyclic ring or heterocyclic ring containing 1 to 3 heteroatoms independently selected from O, N and S,
wherein the carbocyclic or heterocyclic ring is unsubstituted or substituted with 1 to 5 substituents independently selected from the group consisting of halogen, C₁₋₆-alkyl and halo-C₁₋₆-alkyl;
with the proviso that A is not an unsubstituted or substituted pyrazole ring.

5. The compound of at least one of claims 1 to 4, wherein A is unsubstituted or substituted with 1 to 7 substituents independently selected from the group consisting of halogen, OH, CN, C₁₋₆-alkyl, C(O)OR^{a}, OC(O)R^{a}, S(O)—C₁₋₆-alkyl, S(O)₂—C₁₋₆-alkyl, S(O)₂N(R^{a})₂, NR^{a}S(O)₂—C₁₋₆-alkyl and C₃₋₆-cycloalkyl,
wherein the alkyl and cycloalkyl are unsubstituted or substituted with 1 to 3 substituents independently selected from the group consisting of halogen, C₁₋₃-alkyl, halo-C₁₋₃-alkyl, OH, CN and oxo; and
R^{a} is independently hydrogen or C₁₋₆-alkyl.

6. The compound of at least one of claims 1 to 5, wherein A is substituted with 1 to 5 substituents independently selected from halogen, C₁₋₆-alkyl, C₁₋₆-haloalkyl and C₃₋₆-cycloalkyl
wherein cycloalkyl is unsubstituted or substituted with C₁₋₃-alkyl.

7. The compound of at least one of claims 1 to 5, wherein
A is 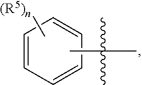
wherein
R⁵ is independently selected from halogen, OH, CN, C₁₋₆-alkyl, C(O)OR^{a}, OC(O)R^{a}, S(O)—C₁₋₆-alkyl, S(O)₂—C₁₋₆-alkyl, S(O)₂N(R^{a})₂, NR^{a}S(O)₂—C₁₋₆-alkyl or C₃₋₆-cycloalkyl,
wherein the alkyl and cycloalkyl are unsubstituted or substituted with 1 to 3 substituents independently selected from the group consisting of halogen, C₁₋₃-alkyl, halo-C₁₋₃-alkyl, OH, CN and oxo;
R^{a} is independently hydrogen or C₁₋₆-alkyl, and
n is 0 to 5.

8. The compound of claim 7, wherein n is 1 to 5 and R⁵ is independently selected from halogen, C₁₋₆-alkyl, C₁₋₆-haloalkyl, and Cₘ-cycloalkyl which is unsubstituted or substituted with C₁₋₃-alkyl.

9. The compound of at least one of claims 1 to 8, wherein
A is 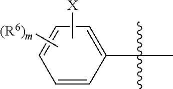
wherein
X is halogen, C₁₋₆-alkyl, C₁₋₆-haloalkyl, or C₃₋₆-cycloalkyl;
R⁶ is halogen; and
m is 0 to 4.

10. The compound of at least one of claims 1 to 9, wherein
A is 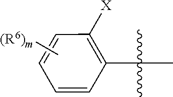
X is halogen, CH₃, CHF₂ or CF₃;
R⁶ is halogen; and
m is 0 to 4.

11. The compound of at least one of claims 1 to 10, wherein B is a 5- or 6-membered heteroaryl containing 1 to 4 heteroatoms independently selected from N, O and S, which is unsubstituted or substituted with 1 to 5 substituents independently selected from the group consisting of halogen, OH, CN, C₁₋₆-alkyl, O—C₁₋₆-alkyl, C(O)OR^{a}, OC(O)R^{a}, S(O)—C₁₋₆-alkyl, S(O)₂—C₁₋₆-alkyl, N(R^{a})₂, C(O)N(R^{a})₂, S(O)₂N(R^{a})₂ and C₃₋₆-cycloalkyl,
wherein the alkyl and cycloalkyl are unsubstituted or substituted with 1 to 3 substituents independently selected from the group consisting of halogen, C₁₋₃-alkyl, halo-C₁₋₃-alkyl, OH, CN and oxo; and
R^{a} is independently hydrogen or C₁₋₆-alkyl.

12. The compound of at least one of claims 1 to 11, wherein B is unsubstituted or substituted with 1 or 2 substituents independently selected from the group consisting of C₁₋₆-alkyl, C₁₋₆-haloalkyl and C₃₋₆-cycloalkyl.

13. The compound of at least one of claims 1 to 12, wherein B is

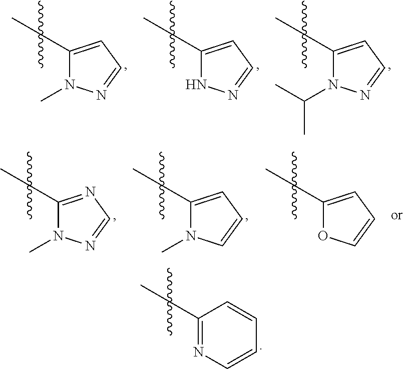

14. The compound of at least one of claims 1 to 13, wherein B is

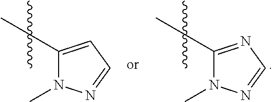

15. The compound of at least one of claims 1 to 10, wherein B is

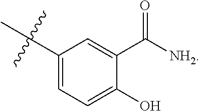 [SMILES: *c1ccc(O)c(C(N)=O)c1]

16. The compound of at least one of claims 1 to 15, wherein each of R¹, R², R³ and R⁴ are hydrogen.

17. The compound of claim 1 or 2, which is selected from the following group consisting of

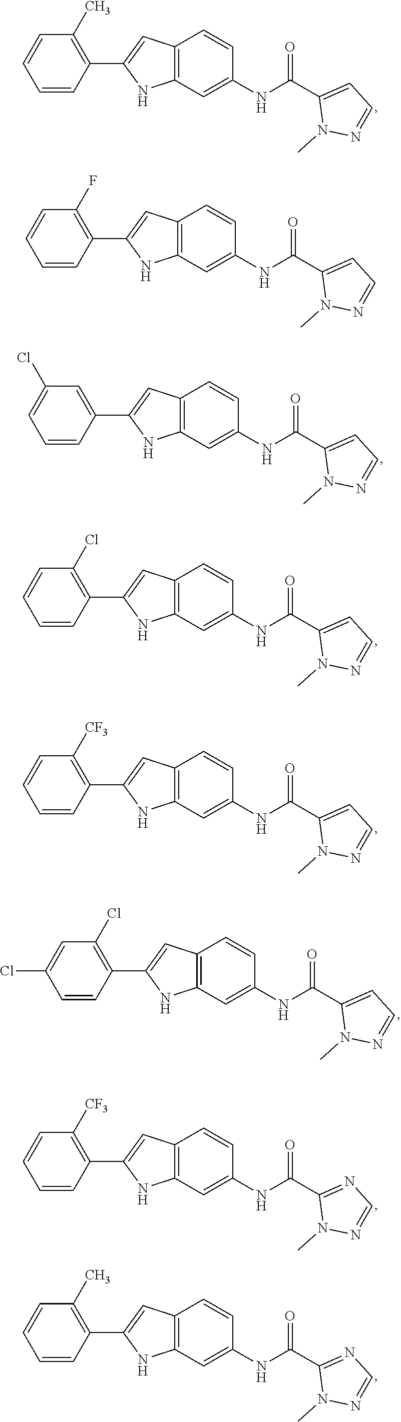

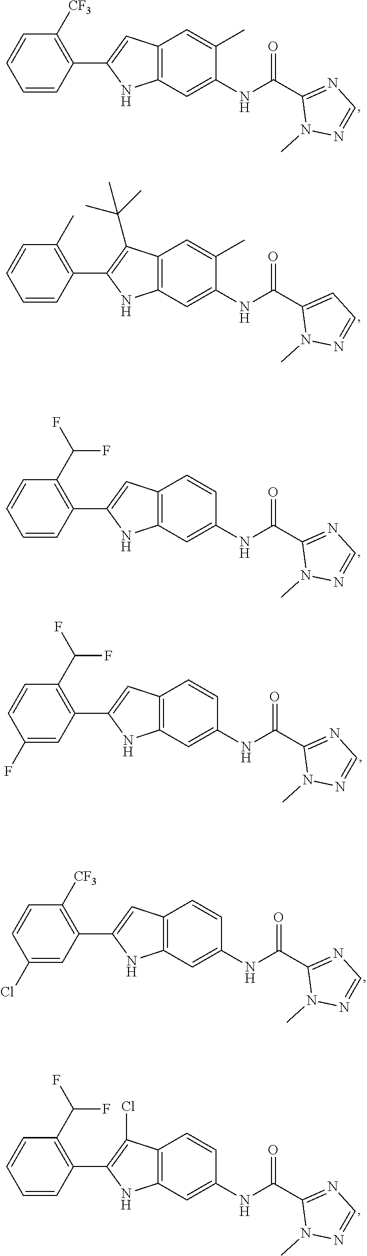 [SMILES: Cc1cc2cc(-c3ccccc3C(F)(F)F)[nH]c2cc1NC(=O)c1ncnn1C ; Cc1cc2c(C(C)(C)C)c(-c3ccccc3C)[nH]c2cc1NC(=O)c1ccnn1C ; Cn1ncnc1C(=O)Nc1ccc2cc(-c3ccccc3C(F)F)[nH]c2c1 ; Cn1ncnc1C(=O)Nc1ccc2cc(-c3cc(F)ccc3C(F)F)[nH]c2c1 ; Cn1ncnc1C(=O)Nc1ccc2cc(-c3cc(Cl)ccc3C(F)(F)F)[nH]c2c1 ; Cn1ncnc1C(=O)Nc1ccc2c(Cl)c(-c3ccccc3C(F)F)[nH]c2c1]

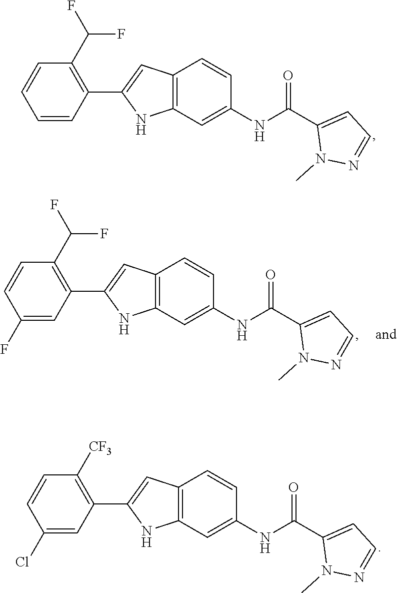 [SMILES: Cn1nccc1C(=O)Nc1ccc2cc(-c3ccccc3C(F)F)[nH]c2c1 ; Cn1nccc1C(=O)Nc1ccc2cc(-c3cc(F)ccc3C(F)F)[nH]c2c1 ; Cn1nccc1C(=O)Nc1ccc2cc(-c3cc(Cl)ccc3C(F)(F)F)[nH]c2c1]

18. A pharmaceutical composition comprising the compound of at least one of claims 1 to 17 and a physiologically acceptable excipient.

19. The compound according to at least one of claims 1 to 17 for use as a medicament.

20. The compound of according to at least one of claims 1 to 17 or the pharmaceutical composition according to claim 18 for use in the prophylaxis and/or treatment of a disease or condition mediated by aryl hydrocarbon receptor (AhR).

21. The compound or pharmaceutical composition for use according to claim 20, wherein the disease or condition mediated by aryl hydrocarbon receptor (AhR) is cancer.

22. The compound for use according to claim 19, wherein the compound is administered with one or more therapeutic agents for cancer selected from the group consisting of PD-1 agent, PD-L1 agent, CTLA-4 agent, IDO1 inhibitor, chemotherapeutic agent, anticancer vaccine, and cytokine therapy, or wherein the compound is administered under irradiation therapy.
